(19) United States
(12) Patent Application Publication
(10) Pub. No.: US 2024/0156682 A1
(43) Pub. Date: May 16, 2024

(54) Reinforced Gas Permeable Blood Storage Bags, and Methods of Preparation Thereof

(71) Applicant: Hemanext Inc., Lexington, MA (US)

(72) Inventors: Robert HARHEN, Haverhill (OM); Peter PIGNONE, Framingham, MA (US); Narendran RENGANATHAN, Plano, TX (US); Jancarlo SARITA, Lynn, MA (US); Jeffrey Karl SUTTON, Medway, MA (US); Michael WOLF, Brookline, MA (US); Michael R. ZOCCHI, Arlington, MA (US)

(73) Assignee: Hemanext Inc., Lexington, MA (US)

(21) Appl. No.: 18/452,051

(22) Filed: Aug. 18, 2023

Related U.S. Application Data
(63) Continuation of application No. 15/758,785, filed on Mar. 9, 2018, now abandoned.
(60) Provisional application No. 62/385,116, filed on Sep. 8, 2016.
(60) Provisional application No. 62/216,774, filed on Sep. 10, 2015.

(51) Int. Cl. A61J 1/10; A01N 1/02; A61J 1/14; A61M 1/02; A61M 1/34; B01D 63/08; B01D 67/00; B01D 69/02; B01D 69/06; B01D 69/10; B01D 71/24; B01D 71/70

(52) U.S. Cl. CPC A61J 1/10; A01N 1/0263; A61J 1/1468; A61M 1/0272; A61M 1/3403; B01D 63/089; B01D 67/0086; B01D 69/02; B01D 69/061; B01D 69/1071; B01D 71/24; B01D 71/701; A61M 2202/0208; A61M 2205/7536; B01D 2323/42; B01D 2325/04; B01D 2325/08; B01D 2325/20

(57) ABSTRACT

The present disclosure relates to improved collapsible blood containers comprising reinforced silicone for use in Oxygen Reduction Disposable kits (ORDKit), devices and methods. The improved collapsible blood containers and methods for the collection of blood and blood components provide for improved burst, tear, and puncture resistance.

### CROSS REFERENCE TO RELATED APPLICATION

This application is a continuation of U.S. application Ser. No. 15/758,785, filed Mar. 9, 2018, which is a U.S. National Stage filing under 35 U.S.C. § 371 of International Application No. PCT/US2016/051115, filed Sep. 9, 2016, which claims benefit of, and priority to, U.S. Provisional Application No. 62/216,774, filed Sep. 10, 2015, and U.S. Provisional Application No. 62/385,116, filed Sep. 8, 2016, each of which are hereby incorporated by reference in their entireties.

### FIELD OF THE INVENTION

The present disclosure relates to Oxygen Reduction Disposable kits (ORDKit), devices and methods for the improved preservation of whole blood and blood components. More particularly, the disclosure relates to the improved devices and methods for the collection of blood and blood components to provide whole blood and blood components having reduced levels of oxygen. The methods, devices and kits of the present disclosure provide for improved quality of blood and blood components for transfusion and improved patient safety and outcome.

### BACKGROUND OF THE INVENTION

U.S. Provisional Application No. 62/131,130, filed Mar. 10, 2015, relates to Oxygen Reduction Disposable kits (ORDKit) (“the '130 Provisional”), devices and methods for the improved preservation of whole blood and blood components and U.S. Provisional Appln. No.: 62/151,957, filed Apr. 23, 2015, relates to improved anaerobic storage bags (ASB) and methods for the improved preservation of whole blood and blood components (“the '957 Provisional”). Both applications are hereby incorporated by reference in their entireties.

Among the devices and methods provided by the '130 and '957 Provisionals are those having collapsible blood containers 102 (and collapsible blood containers 202) made of silicone.

Gas permeable blood bags made with thin (20-50 μm) silicone sheets are structurally weak. To ensure rapid gas depletion during processing, blood containers for oxygen depletion are designed to be thin, leading to structural weakness. Because blood bags must survive rough handling, bags must be structurally robust while still maintaining gas permeability. Blood bags need to be able to survive impacts without leaking or rupturing when dropped. Blood bags further need to be puncture and tear resistant.

Typical methods used in the past to reinforce gas permeable materials utilize rigid frames or support structures, such as perforated metal or plastic, which add cost and also detract from the pliability of the membrane. Such approaches tend to be incompatible with existing blood collection methodologies and require significantly more storage space than the accepted collapsed blood collection kits.

Suitable thin, silicone membranes for use in blood gas depletion devices are not commercially available. In the past, typical methods used to reinforce silicone sheets utilized embedded glass fibers, which detract from the gas permeability. Previous silicone sheets used to prepare reinforced sheets were 150 μm or greater in thickness. While both are more resistant to structural failure and also easier to reinforce, such thick reinforced sheets did not provide the desired oxygen reduction rates. Preparing silicone sheets and collapsible blood containers having silicone thicknesses ranging from 14 μm to 100 μm posed challenges including bursting when handling, unwanted adhesion and cohesion, and puncturing. Further, ISO 3826-1:2013 requires that plastic collapsible blood containers shall not show leakage when placed between two plates and subjected to an internal pressure of 50 kPa above atmospheric pressure for 10 minutes. To solve these problems, gas permeable sheets of silicone sheet have been reinforced with plastic mesh or fabrics to provide the necessary structural integrity for the gas permeable collapsible blood containers. Such reinforced silicone sheets provide for the preparation of collapsible blood containers that when filled with liquid can survive drops of up to 6 feet and are resistant to tearing and punctures.

### SUMMARY OF THE INVENTION

The present disclosure provides for, and includes, an oxygen depletion device for depleting oxygen from blood prior to anaerobic storage comprising an outer receptacle substantially impermeable to oxygen, an inner collapsible blood container formed from a fabric reinforced silicone membrane 600 or a reinforced silicone membrane 700 comprising one or more chambers that are permeable to oxygen, and an oxygen sorbent situated within said outer receptacle.

The present disclosure provides for, and includes, a reinforced silicone membrane 600 comprising a silicone membrane 113 of between 5 μm and 100 μm and a fabric layer of between 50 μm to 1.5 mm thick bonded to one side of said silicone membrane layer 113.

The present disclosure provides for, and includes, a reinforced silicone membrane 700 comprising a silicone membrane 113 having an area 702 and an average thickness 703 of less than 100×10⁻⁶M (μm), and a feature 701 on at least one side of said silicone membrane 113 comprising silicone having an average length 713 of between 100 μm and 10000 μm perpendicular to an average length 714 of between 20 μm and 5000 μm.

The present disclosure provides for, and includes, an injection molded collapsible blood container 102 comprising a silicone membrane 700 comprising a silicone membrane 113 having an area 702 and an average thickness 703 of less than 100×10⁻⁶ M (μm) and a feature 701 on at least one side of the silicone membrane 113 having an average length 713 of between 100 μm and 10000 μm perpendicular to an average length 714 of between 20 μm and 5000 μm.

The present disclosure provides for, and includes, a method of manufacturing a silicone membrane 700 comprising (i) preparing silicone membrane 113 having a thickness of less than 100×10⁻⁶ M (μm); (ii) applying a raised feature 701 having an average length 713 of between 100 μm and 10000 μm perpendicular to an average length 714 of between 20 μm and 5000 μm to the surface of the silicone membrane 113; and (iii) curing the silicone membrane 700.

The present disclosure provides for, and includes, a method of manufacturing a silicone membrane 700, comprising injection molding a silicone membrane 113 having an area 702 and an average thickness 703 of less than 100×10⁻⁶ M (μm) and a feature 701 on at least one side of the silicone membrane 113 having an average length 713 of between 100 μm and 10000 μm perpendicular to an average length 714 of between 20 μm and 5000 μm.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, makes apparent to those skilled in the art how aspects of the disclosure may be practiced.

FIG. 1 illustrates an exemplary embodiment of an oxygen reduction disposable storage system having a blood depletion device having two reinforced silicone collapsible blood containers 102, respectively, and an anaerobic storage bag having a reinforced silicone collapsible blood container 202 according to the present disclosure.

FIGS. 2A to 2E illustrate exemplary structures of an enlarged cross-section view of reinforced silicone membranes 600 according to the present disclosure. FIG. 2B includes an enlarged (left) and further enlarged (right) view of a cross-section of reinforced silicone membrane 600 according to the present disclosure.

FIG. 3 illustrates an exemplary embodiment of a reinforced silicone collapsible blood container 102 according to the present disclosure having a reinforced silicone membrane 600 or a reinforced silicone membrane 700.

FIG. 4 illustrates an exemplary embodiment of a reinforced silicone collapsible blood container 102 according to the present disclosure having a reinforced silicone membrane 600 or a reinforced silicone membrane 700.

FIG. 5 illustrates an exemplary embodiment of a reinforced silicone collapsible blood container 102 according to the present disclosure having a reinforced silicone membrane 600 or a reinforced silicone membrane 700.

FIG. 6 illustrates a reinforced silicone membrane 700 having a raised feature 701, an open area 702, a thickness 703, and having a pattern 722 according to the present disclosure.

FIGS. 7A to 7I illustrate exemplary embodiments of cross sections of raised features 701 according to the present disclosure having a height of length 714 and a width of length 713.

FIGS. 8A to 8N illustrate exemplary patterns 720 of raised features 701 of a reinforced silicone membrane 700 according to the present disclosure.

FIGS. 9A to 9D illustrate an exemplary embodiment of a reinforced silicone collapsible blood container 102 prepared using an injection molding process comprising a reinforced silicone membrane 700 having a raised feature 701, an open area 702, a thickness 703, and having a pattern 724 according to the present disclosure.

FIGS. 10A to 10C illustrate an exemplary embodiment of a reinforced silicone membrane 700 and a collapsible blood container 202 prepared using a reinforced silicone membrane 700. FIG. 10A shows a reinforced silicone membrane 700 having a raised feature 701 having a pattern 721, and an open area 702. FIG. 10B shows a reinforced silicone membrane 700 of FIG. 10A having a cross section 761, a height of length 714, and a width of length 713. FIG. 10C presents a collapsible blood container 102 prepared from a reinforced silicone membrane 700 of FIG. 10A.

FIGS. 11A and 11B illustrate exemplary embodiments of a reinforced silicone membrane 700 prepared by compression molding and by injection molding respectively according to the present disclosure.

FIGS. 12A to 12D illustrate exemplary embodiments of roller dies suitable for use in the manufacture of a reinforced silicone membrane 700 using a calendaring method according to an aspect of the present disclosure.

FIG. 13 illustrates an exemplary embodiment of an automated method of manufacturing reinforced silicone collapsible blood containers according to the present disclosure.

FIG. 14 illustrates an exemplary embodiment of an automated method of manufacturing reinforced silicone collapsible blood containers using a continuous process according to the present disclosure.

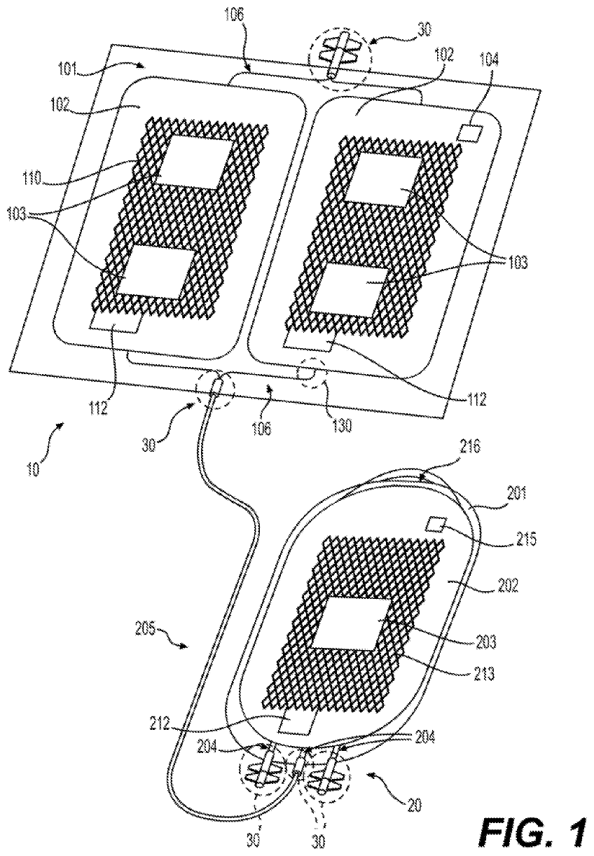

Corresponding reference characters indicate corresponding parts throughout the several views. The examples set out herein illustrate several embodiments of the invention but should not be construed as limiting the scope of the invention in any manner.

### DETAILED DESCRIPTION

To address such needs and others, the present disclosure includes and provides devices and methodology for the preservation of blood and blood components in which the preparation of oxygen reduced blood and blood components is initiated at the donor collection stage.

Before explaining at least one aspect of the disclosure in detail, it is to be understood that the disclosure is not necessarily limited in its application to the details set forth in the following description or exemplified by the Examples. The disclosure is capable of other aspects or of being practiced or carried out in various ways.

As used herein, the term “bag” refers to collapsible containers prepared from a flexible material and includes pouches, tubes, and gusset bags. As used herein, and included in the present disclosure, the term includes folded bags having one, two, three, or more folds and which are sealed or bonded on one, two, three, or more sides. Bags may be prepared using a variety of techniques known in the art including bonding of sheets of one or more materials. Methods of bonding materials to form bags are known in the art. Also included and provided for in the present disclosure are containers prepared by injection and blow molding. Methods to prepare blow molded and injection molded containers are known in the art. Preferred types of blow molded or injection molded containers are flexible containers that can be reduced in size for efficient packing and shipping while being capable of expanding to accommodate blood or blood components for reduction of oxygen. They also may be designed to conform to the volume of the blood until they are fully expanded. As used throughout the present disclosure, the bags are a form of collapsible container and the two terms are used interchangeably throughout the present disclosure.

As used herein, the term “collapsible container” includes bags, containers, enclosures, envelopes, pouches, pockets, receptacles, and other devices that can contain and retain a liquid or fluid. In certain aspects, the collapsible container may be manufactured by conventional means such as injection molding or insert molding. In other aspects, the collapsible container may be prepared from sheets of polymer materials that are bonded together using methods known in the art to prepare containers capable of holding a volume. Such collapsible containers are well known in the art. See, for example, U.S. Pat. No. 3,942,529 issued to Waage; U.S. Pat. No. 4,131,200 issued to Rinfret; and U.S. Pat. No. 5,382,526 issued to Gajewski et al. Suitable methods for bonding polymer materials to prepare collapsible containers according to the present disclosure include heat welding, ultrasonic welding, radio frequency (RF) welding, and solvent welding. In certain aspects, multiple bonding methods may be used to prepare collapsible containers according to the present disclosure. Collapsible container according to the present disclosure include enclosures having one or more pleats, folds, diaphragms, bubbles, and gussets. Methods for preparing collapsible containers are known in the art. See, for example, U.S. Pat. No. 3,361,041 issued to Grob; U.S. Pat. No. 4,731,978 issued to Martensson; U.S. Pat. No. 4,998,990 issued to Richter et al.; and U.S. Pat. No. 4,262,581 issued to Ferrell. Also included and provided for in the present disclosure are containers having combinations of both flexible and inflexible parts, wherein the flexible parts allow for the expansion of the volume through, for example, pleats, folds or gussets and other similar geometric features in the packaging shape, whereas the inflexible parts may provide rigidity and geometry definition to the container. Methods and designs for preparing collapsible containers having both flexible and inflexible parts are known in the art, such as described by Randall in U.S. Pat. No. 6,164,821 and by LaFleur in U.S. Pat. No. 5,328,268.

As used herein the term “about” refers to ±10%.

The terms “comprises,” “comprising,” “includes,” “including,” “having,” and their conjugates mean “including but not limited to.”

The term “consisting of” means “including and limited to.”

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a,” “an,” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3,” “from 1 to 4,” “from 1 to 5,” “from 2 to 4,” “from 2 to 6,” “from 3 to 6,” etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

As used herein the term “method” refers to manners, means, techniques, and procedures for accomplishing a given task including, but not limited to, those manners, means, techniques, and procedures either known to or readily developed from known manners, means, techniques, and procedures by practitioners of the chemical, pharmacological, biological, biochemical, and medical arts.

The present disclosure provides for, and includes, an oxygen depletion device 10 for depleting oxygen from blood comprising an outer receptacle 101 substantially impermeable to oxygen, inner collapsible blood container 102 that is permeable to oxygen, and an oxygen sorbent 103 situated within outer receptacle 101. As provided herein, an inner collapsible blood container 102 is prepared from reinforced silicone membranes 600 or reinforced silicone membranes 700, or combinations of the each. The present disclosure also provides for the manufacture of reinforced silicone membranes 600 or reinforced silicone membranes 700.

The present disclosure also provides for, and includes, oxygen depletion devices 10 configured to be a blood collection and oxygen depletion device 10. Oxygen depletion devices configured to collect and reduce blood oxygen differ from the oxygen depletion device 10 as described throughout this specification in that a blood collection and oxygen depletion device 10 further includes an anticoagulant to prevent coagulation of the whole blood during the collection process. In certain aspects, the anticoagulant solution of a blood collection and oxygen depletion device 10 is provided in the blood collection and oxygen depletion device 10. Accordingly, included anticoagulant solutions are also oxygen depleted anticoagulant solutions. In the alternative, anticoagulant solutions may be included separately, either as oxygen depleted solutions or solutions having oxygen. A blood collection and oxygen depletion device 10 is intended to be used with whole blood collected from a donor. As used throughout the present disclosure, the oxygen and depletion device 10 includes and provides for blood collection and oxygen depletion device 10. The two terms can be, and are, used interchangeably.

As used herein, the outer receptacles are prepared from materials that are substantially impermeable to oxygen and optionally impermeable to carbon dioxide. In certain aspects, an outer receptacle 101 is prepared from flexible membrane materials. In other aspects, an outer receptacle 101 is prepared from a stiff, or inflexible membrane material.

The present disclosure provides for, and includes, an outer receptacle 101 substantially impermeable to oxygen. As used herein, an outer receptacle 101 that is substantially impermeable to oxygen is sufficiently impermeable to oxygen to allow no more than 10 cc of oxygen inside the receptacle over a period of 3 months, and more preferably no more than 5 cc of oxygen over 6 months. As used herein, the term substantially impermeable to oxygen (SIO) refers to materials and compositions that provide a barrier to the passage of oxygen from one side of the barrier to the other, sufficient to prevent significant increases in the partial pressure of oxygen. Outer receptacles 101 as used herein are described in detail in International Application Nos. PCT/US2016/02179 and PCT/US2016/029069.

The present disclosure provides for, and includes, the preparation of outer receptacles 101 and inner collapsible blood container 102 from a membrane or film. As used herein, membranes generally refer to materials used to prepare an inner collapsible blood container 102 and films are used to refer to materials used to prepare outer receptacle 101. A membrane comprises one or more layers of materials in the form of a sheet that allows or prevents one or more substances to pass through from one side of the sheet to the other side of the sheet. As used herein, membranes may also be prepared as tubes suitable for connecting together components of oxygen depletion devices 10, blood collection kits, or connecting together elements of blood collection devices, additive solution bags, leukocyte reduction filters, and anaerobic storage bags. As used throughout, it is understood that a membrane of the present disclosure may be formed as a sheet or a tube depending on the application. Also as previously provided, membranes to prepare outer receptacles 101 are substantially impermeable to oxygen while an inner collapsible blood container 102 is permeable to oxygen.

As used herein, an inner collapsible blood container 102 is permeable to oxygen. In certain aspects, an inner collapsible blood container 102 is permeable to oxygen and carbon dioxide. In other aspects, an inner collapsible blood container 102 is permeable to oxygen and impermeable to carbon dioxide. Similarly, as used herein, reinforced silicone membranes 600 or reinforced silicone membranes 700 are permeable to oxygen. In other aspects, reinforced silicone membranes 600 or reinforced silicone membranes 700 are permeable to both oxygen and carbon dioxide. Unless specifically provided, the reinforced silicone membranes 600 or reinforced silicone membranes 700 are essentially impermeable to liquid water.

Membrane permeation flux, for a gas, is defined as the volume flowing through the membrane per unit area per unit time. The SI unit used is m³/m²·s. For gases and vapors, the volume is strongly dependent on pressure and temperature. Accordingly, permeation fluxes for gases are often given in terms of standard temperature and pressure (STP) which is defined as 0° C. and 1 atmosphere (1.0013 bar) (e.g., 273° K and 760 torr). As noted above, the rate of passage depends on a driving force or difference between the two sides of the membrane, and this dependence is incorporated in the permeability coefficient, P, or simply the permeability.

Permeability (P) is defined as the permeability flux per unit of driving force per unit of membrane thickness. The SI unit for the permeability coefficient P is provided in Table 1. A common unit for gas separation, as in the present disclosure, is the Barrer and is also presented in Table 1. The term cm³ gas (STP)/cm³s refers to the volumetric trans-membrane flux of the diffusing species in terms of standard conditions of 0° C. and 1 atmosphere pressure, the term cm refers to the membrane thickness, and cm-Hg refers to the trans-membrane partial pressure driving force for the diffusing species. Permeability must be experimentally determined.

TABLE 1
Permeability Units

| | Units of Permeability |
|---|---|
| “Volumetric” permeability | $1 Barrier = \frac{\begin{matrix}1 {0}^{- 10} \cdot {cm}^{3} gas ( STP ) \cdot \\ ( cm membrane thickness )\end{matrix}}{( {cm}^{2} membrane area ) \cdot s \cdot ( cmHg pressure )}$ |
| “Molar” permeability | $\frac{mol}{m \cdot Pa \cdot s} ( SI units ) = \frac{\begin{matrix}( {mol}_{i} permeating ) \cdot \\ ( m membrane thickness )\end{matrix}}{\begin{matrix}( {m}^{2} membrane area ) \cdot \\ s \cdot ( Pa pressure )\end{matrix}}$ |

Membranes suitable for the methods and devices according to the present disclosure include dense membranes, porous membranes, asymmetric membranes, and composite membranes. Dense membranes are membranes prepared from solid materials that do not have pores or voids. Materials permeate dense membranes by processes of solution and diffusion. Examples of dense membranes include silicone membranes (polydimethyl siloxane, or PDMS).

The present disclosure provides for, and includes, inner collapsible blood containers 102 prepared from membranes 113 that are characterized primarily by their permeability to oxygen. Unless indicated otherwise, a “substantially impermeable membrane” refers to membranes that are substantially impermeable to oxygen. However, in certain devices and methods, the membranes may be further characterized by the permeability or impermeability to carbon dioxide. For certain applications, the membrane material is substantially impermeable to oxygen and provides a barrier to the introduction of oxygen to the blood, blood component, or a blood collection kit comprised of multiple components. Such substantially impermeable membranes are generally used to prepare outer receptacles of the present disclosure. Suitable substantially impermeable membranes may also be used to prepare tubing for connective components of the devices and kits. Substantially impermeable membranes may comprise a monolayer or be laminated sheets or tubes having two or more layers.

The present disclosure also provides for, and includes, membranes 113 that are substantially permeable to oxygen. Membranes 113 that are substantially permeable to oxygen are used in the present disclosure for the preparation of inner collapsible blood containers 102. In certain aspects, the membranes 113 that are permeable to oxygen are also biocompatible membranes, approved and suitable for extended contact with blood that is to be transfused into a patient. Like substantially impermeable membranes, substantially permeable membranes 113 may comprise a monolayer or may comprise a laminated structure having two or more layers. As provided herein, membranes 113 that are substantially permeable to oxygen are membranes 113 suitable for use in the preparation of reinforced silicone membranes 600 or reinforced silicone membranes 700. Accordingly, except as modified to provided fabric reinforcement described below and throughout the specification as membranes 600, or modified to provided silicone reinforcement described below and throughout the specification as membranes 700, the permeability and other features of the membranes 113 (or 114) are retained throughout.

In an aspect, oxygen permeable membranes 113 having a permeability to oxygen of greater than about 2.5×10⁻⁹ cm³ O₂ (STP)/((cm² s)*(cm Hg cm⁻¹) is used for the preparation of a collapsible blood container 102. In another aspect, oxygen permeable membranes 113 having a permeability to oxygen greater than about 5.0×⁻⁹ cm³ O₂ (STP)/((cm 2 s)*(cm Hg cm⁻¹) is used for the preparation of a collapsible blood container 102. In yet another aspect, oxygen permeable membranes 113 have a permeability to oxygen of greater than about 1.0×10⁻⁸ cm³ O₂ (STP)/((cm² s)*(cm Hg cm⁻¹). In certain aspects, oxygen permeable membranes 113 suitable for use in the preparation of a collapsible blood container 102 are characterized by a Barrer value of greater than about 25. In other aspects, oxygen permeable membranes 113 suitable for use in the preparation of a collapsible blood container 102 are characterized by a Barrer value of greater than about 50. In certain other aspects, oxygen permeable membranes 113 suitable for use in the preparation of a collapsible blood container 102 are characterized by a Barrer value of greater than about 100.

In an aspect, a membrane 113 that is substantially permeable to oxygen can be dense membranes prepared from non-porous materials. Examples of suitable materials that are capable of high oxygen permeability rates include silicones, polyolefins, epoxies, and polyesters.

In aspects according to the present disclosure, a membrane 113 suitable for use in preparing an inner collapsible blood container 102 is a reinforced silicone membrane that is less than 100 μm thick and greater than 10 μm.

The present disclosure provides for, and includes, preparing membranes 113 that are substantially permeable to oxygen, not only by selecting the material, but also by selecting and controlling the thickness. As provided above, permeability is proportional to the thickness of the membrane. Accordingly, improved permeability may be achieved by decreasing the thickness of the membrane. In certain aspects, the minimum thickness is determined by its strength and resistance to puncture and tearing.

The present disclosure also provides for, and includes, membranes 113 that are substantially permeable to oxygen that are prepared using blow molding and injection molding techniques. Suitable materials for preparing inner collapsible blood containers 102 using blow molding and injection molding include silicone materials such as Bluestar 4350, 50 durometer, Silbione grade liquid silicone rubber and Shin-Etsu KEG-2000-40A/B Liquid Silicone. The silicone durometer choice is carefully chosen for collapsibility and permeability, followed by a well controlled wall thickness. Thinner materials will have a higher permeability and are less able to withstand puncturing, tearing, and dropping when formed into a container and filled with liquid. Methods to prepare blow molded and injection molded collapsible blood containers 102 are known in the art, for example, U.S. Pat. No. 4,398,642 issued to Okudaira et al.; U.S. Pat. No. 7,666,486 issued to Sato et al.; U.S. Pat. No. 8,864,735 issued to Sano et al.; and U.S. Patent Application Publication No. 2012/0146266 by Oda et al. In an aspect, a blow molded collapsible blood container 102 can be prepared using LDPE used in the manufacture of collapsible water containers. As provided below, suitable blow molded or injection molded collapsible blood containers 102 have a permeability to oxygen of at least about 25 Barrer.

In an aspect according to the present disclosure, the collapsible blood container 102 can be manufactured from microporous membrane 113 by various sealing methods such as heat sealing, thermal staking, and adhesive bonding. In one aspect according to the present disclosure, a pair of PVDF microporous membranes are bonded together around the periphery with a section of PVC inlet tubing in place in the seam using an adhesive such as Loctite 4011 in conjunction with an adhesive primer such as Loctite 770. In another aspect according to the present disclosure, a collapsible blood container can be manufactured from a pair of microporous membranes by heat sealing the 3 or 4 edges of the pair of membranes together with a section of multilayer tubing sealed into the seam to provide for fluid connectivity.

The present disclosure provides for, and includes, a collapsible blood container 102 that is prepared from more than one type of membrane 113. In an aspect, a collapsible blood container 102 comprises a first membrane 113 and a second membrane 114 suitably bonded to prepare a container. In another aspect, a collapsible blood container 102 comprises a membrane 113 combined with a second membrane 114 that has a permeability of less than about 30% of the permeability of first membrane 113. In certain aspects, a second membrane 114 comprises a membrane that is relatively impermeable or insufficiently permeable to provide sufficient deoxygenation on its own, but can be combined with a suitable membrane 113. In certain aspects, the second membrane 114 is relatively impermeable. In further aspects, the second membrane 114 comprises a molded membrane that incorporates ridges, baffles, or other structures to facilitate mixing. In an aspect, the second membrane 114 may comprise a rigid structure joined to an oxygen permeable membrane 113. In aspects according to the present disclosure, the second membrane 114 is heat sealed to membrane 113.

In certain aspects, the inner collapsible blood container 102 contains flow baffles located internal or external to the blood contact area that provide an increase in the turbulence inside the collapsible blood container 102 when agitated. In an aspect, baffles are located 1 to 2 inches from each other and comprise 10 to 45% of the inner collapsible blood container 102 area. In certain aspects, the flow baffles may comprise raised features 701 of a reinforced membrane 700 that are oriented internally in an inner collapsible blood container 102. Accordingly, raised features 701, suitably configured and placed on the internal surface of an inner collapsible blood container 102 provide an increase in the turbulence of the blood when agitated. When oriented on the internal surface of the inner collapsible blood container 102, features 701 serve to both strengthen the membrane 113 and provide for improved mixing of the blood during deoxygenation.

In other aspects, the flow baffles may comprise fibers 601 of a reinforced membrane 600 that are oriented internally in an inner collapsible blood container 102. Accordingly, raised fibers 601, suitably configured and placed on the internal surface of an inner collapsible blood container 102 provide an increase in the turbulence of the blood when agitated. When oriented on the internal surface of the inner collapsible blood container 102 prepared from a reinforced silicone membrane 600, fibers 601 serve to both strengthen the membrane 113 and provide for improved mixing of the blood during deoxygenation.

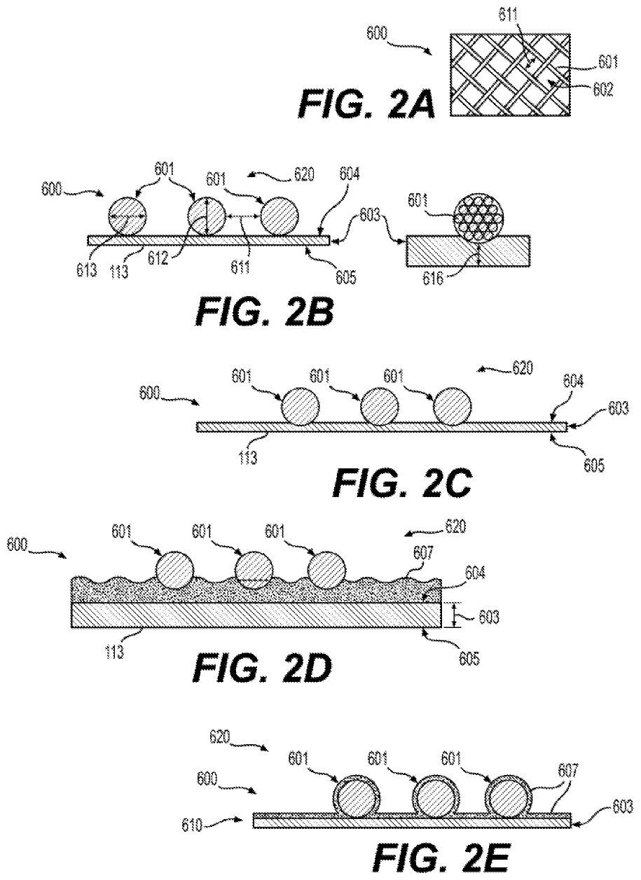

As provided herein, an inner collapsible blood container 102 may be prepared from silicone membrane 113 that has been reinforced with a fabric (“reinforced membrane 600” or “membrane 600”, see FIGS. 2A to 2E). Reinforced membranes 600 provide for the manufacture of inner collapsible blood container 102 that may help comply with ISO standard 3826-1:2013 that requires that plastic collapsible blood containers shall not show leakage when placed between two plates and subjected to an internal pressure of 50 kPa above atmospheric pressure for 10 minutes. When the thickness of a silicone membrane 113 is reduced below about 100 μm, the strength of the membrane is significantly compromised.

Non-reinforced silicone membranes 113 are unsuitable for use in an oxygen depletion device 10 and the inner collapsible blood container 102 because once filled with blood (or liquid generally), they are subject to breakage when dropped. Such reduced thickness silicone membranes 113, while suitable under controlled conditions are unsuitable for use under standard conditions present during blood collection and processing. At the same time, to achieve suitable rates of oxygen depletion in oxygen depletion devices 10 according the present disclosure, thinner thicknesses of silicone are desirable. As provided below, reinforced silicone membranes 113 that are 14 μm, 25 μm, and 50 μm thick when incorporated into an oxygen depletion device 10 provide suitable rates of oxygen transfer.

The present disclosure provides for, and includes, a collapsible blood container 102 that is substantially permeable to oxygen and is a membrane prepared from a reinforced membrane 600. In aspects according the present disclosure, the collapsible blood container 102 can be prepared from a reinforced membrane 600 having a silicone thickness of between 5 μm and 100 μm. As used herein, a thickness of a reinforced membrane 600 refers to the thickness of the permeable membrane. As described below, the reinforcing fabric may be significantly thicker than the permeable membrane it reinforces. Also as used herein, the thickness of a blood container 102 refers to the thickness of the permeable membrane from which it constructed. In other aspects, the collapsible blood container 102 can have a thickness of between 5 μm and 75 μm. In other aspects, the collapsible blood container 102 can have a thickness of between 20 μm and 100 μm. In another aspect the collapsible blood container 102 is between 30 μm and 100 μm thick. In yet another aspect, the collapsible blood container 102 is between 50 μm and 100 μm thick. In a further aspect, the thickness of the collapsible blood container 102 can be between 20 μm and 75 μm. The present disclosure provides for, and includes, a collapsible blood container 102 that is 14 μm in thickness. In another aspect, the collapsible blood container 102 is 25 μm thick. In yet another aspect, the collapsible blood container 102 is 50 μm thick. In an additional aspect, the collapsible blood container 102 is 75 μm thick.

In aspects according the present disclosure, the collapsible blood container 102 can be prepared from a reinforced membrane 600 having a thickness of between 20 μm and 75 μm. In other aspects, the collapsible blood container 102 can have a thickness of between 20 μm and 50 μm. In other aspects, the collapsible blood container 102 can have a thickness of between 40 μm and 75 μm. In another aspect, the collapsible blood container 102 is between 40 μm and 50 μm thick. In yet another aspect, the collapsible blood container 102 can have a thickness of between 20 μm and 30 μm.

Suitable silicone membranes include commercially available membranes and membranes prepared from condensation or addition cured silicones. Non-limiting examples of silicone membranes are available from Wacker Silicones, such as the Silpuran® brand of medical grade silicone sheet membranes (Wacker Silicones, Adrian, MI) and Polymer Sciences PS-1033 P-Derm® silicone elastomer membrane (Polymer Sciences, Inc., Monticello, IN). In an aspect, the silicone membrane may be Polymer Sciences PS-1033 or Silpuran® 6000 silicone. Silicone membranes can be prepared from various liquid silicone rubber (LSR) materials, which are available from a number of silicone suppliers, such as Wacker Silicones (Adrian, MI), Shin-Etsu Silicones of America (Akron, OH), NuSil Technology (Carpenteria, CA), and Blue Star Silicones (East Brunswick, NJ), to name a few.

Two part platinum cure liquid silicone rubber (e.g., condensation cured) and silicone dispersions are suitable for creating thin sheets for medical applications. Liquid silicone rubber (LSR); such as Wacker Silpuran 6000, Shin-Esu KEG2000, Dow Corning QP1, or NuSil MED-4901 is supplied with as a separate “A” component and “B” component that must be thoroughly mixed by the manufacturer's suggested method in order to initiate curing. Silicone dispersions, such as NuSil MED10-6640, are also supplied as an “A” component and “B” component that must be thoroughly mixed in order to initiate curing; however, these components are supplied in a suspension of a solvent, such as xylene. Xylene makes these silicones less viscous, which makes thin sheet fabrication easier. With all of these silicones, curing is accelerated with the application of heat.

One part Silicone RTV (room temperature vulcanizing), such as Wacker Silpuran 4200, can also be used to create sheets. One part silicones of the RTV type cure at ambient temperature using the moisture in air, generally have a long cure time and may not be preferred for large scale manufacturing using the methods described below. In certain aspects, one part silicone RTV can be used to prepare frames 120 as described below suitable for joining a reinforced membrane 600 to prepare inner collapsible blood containers 102. In other aspects, one part silicone RTV can be used to prepare frames 120 as described below suitable for joining a reinforced membrane 700 to prepare inner collapsible blood containers 102. Curing times may also be increased by introducing moist air that can accelerate the curing process.

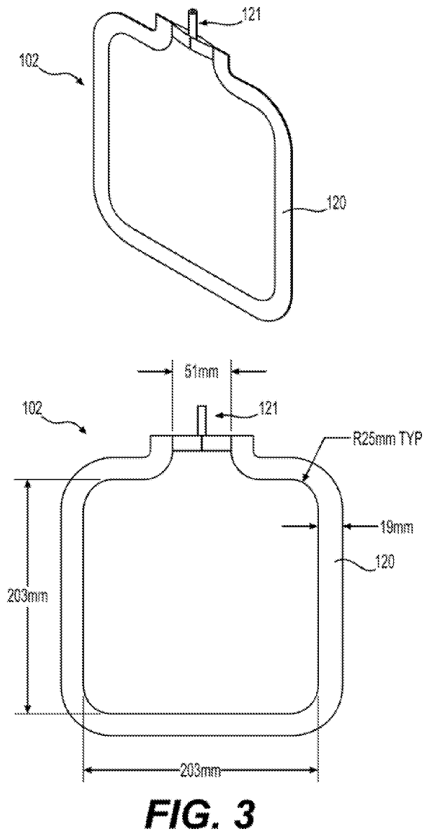

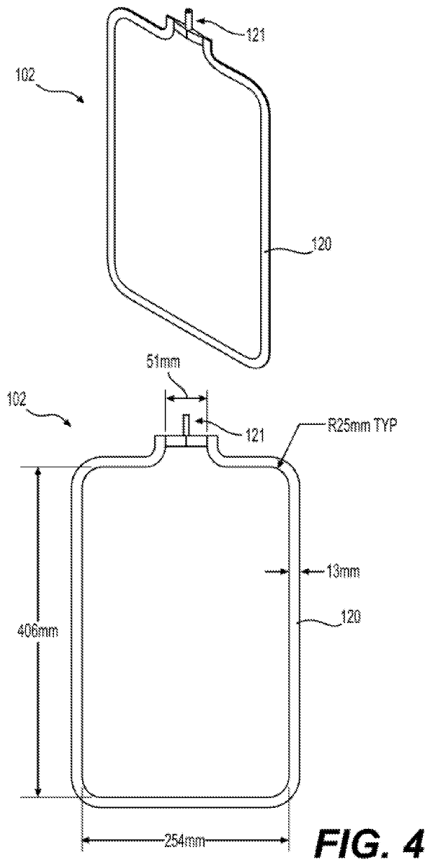

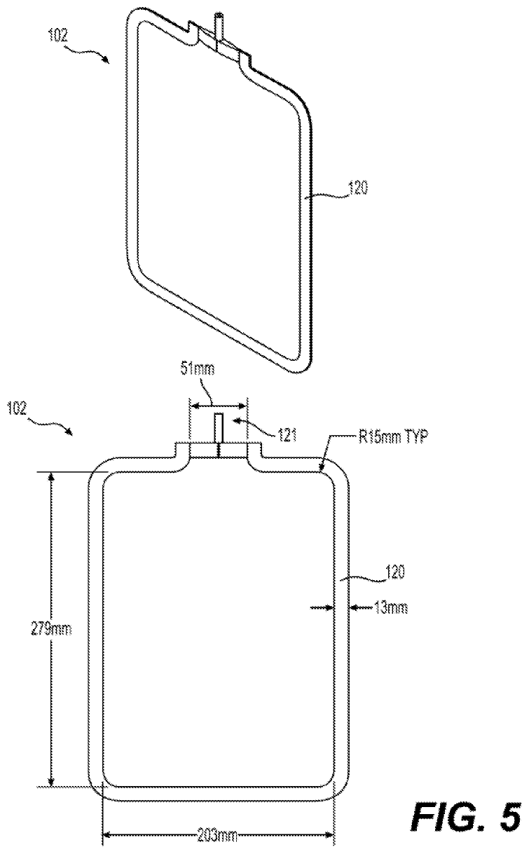

In aspects according to the present disclosure, two part platinum cure high consistency rubber (HCR) silicone, such as NuSil MED-4050 or SIL2-5070, may be used to create a frame 120 that joins two reinforced silicone sheets (reinforced membranes 600 or 700) to create an inner collapsible blood container 102. (See FIGS. 3 to 5, illustrated as item 120). An advantage of HCR is a very high viscosity, improving to manipulability and handling during fabrication. HCR is supplied by the manufacturer in two components that must be thoroughly mixed by the manufacturer's suggested method in order to initiate curing. Heat can be applied to the mixed resin in order to accelerate curing once a container is fabricated. Examples of the preparation of reinforced membrane 600 using heat regimens to partially cure the silicone are provided below. The frame 120 becomes and integral component of the inner collapsible container 102.

A reinforced membrane 600 comprises a thin silicone layer having high oxygen permeability and a fabric reinforcing layer. Reinforced membranes 600 are suitable for the preparation of inner collapsible containers 102 for use in oxygen depletion devices 10. The reinforced silicone membranes are further characterized as having a relatively smooth surface for contact with the blood or blood component for depletion. In certain aspects, additional features may be introduced into the blood contacting surface of a reinforced membrane 600 to provide for additional mixing (i.e., features in addition to fabric 601, that when placed in contact with blood and aid in mixing).

Silicone is generally resistant to attachment to non-silicone materials. Accordingly, the present disclosure provides for, and includes, methods for joining the reinforcing fabric material to the silicone. The resulting reinforced membranes 600 are then used for the production of inner collapsible containers 102. As described below, the process of preparing reinforced membranes 600 and incorporating them into inner collapsible containers 102 can be automated for large scale industrial production, either in batch mode or as continuous production line.

Referring to FIGS. 2A to 2E, the various processes for the preparation of reinforced membranes 600 result in membranes 600 having differing overall geometries. As used throughout, membranes 600 are reinforced membranes 600. Membranes 600 may also include reinforced membranes prepared from other, non-silicone materials. FIG. 2A shows a top view of the reinforcing fabric 600, wherein the fabric has discrete fibers 601 and open areas 602 between the fibers. The fibers 601 are spaced at least about 0.1 mm apart to provide gas permeability of the open areas 602, but no more than about 4 mm apart to provide the reinforcement strength needed.

The present disclosure provides for, and includes a membrane 600 as illustrated in FIG. 2B. Now referring to FIG. 2B, which is a cross-section view of a membrane 600 showing the discrete fibers 601 of a reinforcing fabric 620 and a silicone membrane 603 (e.g., a membrane 113), having an outer surface 604 and an inner surface 605, wherein the reinforcing fabric 620 (comprising fibers 601) is placed on the outer surface 604 of the silicone membrane 603. In an aspect of the invention, the silicone membrane 603 is partially cured and contains a suitable solvent, such as xylene, and has a thickness of about 30-75 μm before complete curing and removal of solvent. In another aspect of the invention, the silicone membrane 603 is fully cured and contains no solvent.

The present disclosure provides for, and includes a membrane 600 as illustrated in FIG. 2C. FIG. 2C is a cross-section view of a membrane 600, having a reinforcing fabric 620 and a silicone membrane 603, showing discrete fibers 601 of the reinforcing fabric 620. The membrane 600 of FIG. 2C is representative of FIG. 2B having a partially cured silicone membrane 603 after pressing the reinforcing fabric 620 into the outer surface 604 of the partially cured silicone membrane 603 and heating the structure to fully cure the silicone membrane 603 and remove the solvent. The discrete fibers 601 are embedded in and attached to the cured silicone membrane 603, but do not protrude through the inner surface 605. In an aspect of the invention, the membrane 600 of FIG. 2C provides a gas permeable material having sufficient strength for routine handling when used in an inner collapsible container 102.

The present disclosure provides for, and includes a membrane 600 as illustrated in FIG. 2D that is a cross section view of a membrane 600, having a reinforcing fabric 620 and a silicone membrane 603, showing discrete fibers 601 of the reinforcing fabric, and further having a bonding layer 607. The bonding layer 607 is comprised of a silicone LSR, and optionally a suitable solvent such as xylene. In aspects according to the present disclosure, the uncured bonding layer 607 is dispensed onto a fully cured silicone membrane 603, such as by spraying or knife coating, to yield a thin layer having a thickness of about 10-50 μm before placing the reinforcing fabric 620 onto the uncured bonding layer 607. The membrane 600 is then heated to about 115-121° C., or according to manufacturer's instructions, to completely cure the bonding layer 607. The fully cured membrane 600 is suitable for use in an inner collapsible container 102.

FIG. 2E is a cross section view of a fabric reinforced silicone membrane 600, having a reinforcing fabric 620 and a silicone membrane 603, showing discrete fibers 601 of the reinforcing fabric, and further having a bonding layer 607. Silicone layer 610 is comprised of silicone membrane 603 and bonding layer 607.

The reinforcing fabric 620 is first placed onto a fully cured silicone membrane 603, and a bonding layer 607 is comprised of an uncured silicone LSR, and optionally a suitable solvent such as xylene is dispensed, such as by spraying, to yield a thin coating having a thickness of about 10-50 μm onto the reinforcing fabric 620 and the fully cured silicone membrane 603. The resulting membrane 600 is then heated to about 115-121° C. to completely cure the bonding layer 607.

In another aspect according to the present disclosure, a reinforcing fabric 620 is dipped in an uncured silicone LSR, and optionally a suitable solvent such as xylene, before placing the reinforcing fabric 620 onto a fully cured silicone membrane 603, followed by curing with heat to yield the structure shown in FIG. 2E.

The present disclosure provides for, and includes, reinforced membranes 600 that are reinforced with a fabric. As used herein, a “fabric” refers to a woven or non-woven fabric or mesh. Also provided for and included in the present disclosure are silicone membranes 113 having fabrics that are configured as a mesh. As used herein, a “mesh” refers to a network of spaces in a net or network comprising a network of cords or threads. In some aspects, a mesh may be a woven cloth or fabric. In other aspects, a mesh may be a nonwoven cloth or fabric. As used herein, fabrics are distinguishable from the meshes used as a spacer 111.

Attaching a reinforcing fabric to a silicone membrane can be achieved by various methods as previously described, including casting, coating, and spot bonding. As noted above, adhesion of silicone to some materials can be low resulting in resistance to attachment of the reinforcing fabric. As provided above, fabrics can be bonded by embedding, partially, or completely the fabric in silicone during manufacture.

The present disclosure provides for and includes methods to enhance the bonding or adhesion of the fabric to the silicone membrane 113 to prepare membranes 600. In an aspect, the adhesion of the fabric to the silicone can be enhanced by plasma treatment of either or both of the materials to be bonded. Plasma treatment for adhesion promotion is well known by one skilled in the art, and suitable processes include vacuum plasma, corona discharge, and atmospheric pressure plasma processing. The plasma treatment of the material before bonding provides for the creation of reactive groups on relatively inert surfaces of materials such as silicones and polyolefins, as well as provides for the removal of surface contaminants from these surfaces. Suitable equipment to treat the materials with atmospheric pressure plasma include the Openair® plasma systems (Plasmatreat USA, Elgin, IL) and the ULD plasma curtain from AcXys Technologies (Le Vinoux, France).

Fabrics for preparing reinforced membranes 600 according to the present disclosure may be prepared from polymers, carbon fibers, fiberglass, natural fibers, and other materials that can be prepared as a mesh. Fabrics may be woven meshes prepared from monofilament synthetic or natural fibers or yarns. In other aspects, woven fabrics may be prepared from multifilament synthetic fibers or yarns.

In an aspect the fabric may be nylon, polybutylene terephthalate (PBT), polyester, polyethylene, polypropylene, polytetrafluoroethylene (PTFE), polypropylene/polyethylene (PP/PE) blends or synthetic yams or fibers. In an aspect, the material for the preparation of fabrics for preparing reinforced membranes 600 is a polyester fabric. In an aspect, the material for the preparation of fabrics for preparing reinforced membranes 600 is a nylon fabric. In an aspect, the material for the preparation of fabrics for preparing reinforced membranes 600 is a polyethylene fabric. Exemplary fabrics suitable for the preparation of a reinforced membrane 600 include polyester fabrics from Textile Development Associates, Surgical Mesh Division. Suitable fabrics include, but are not limited to catalog numbers PETKM2002, PETKM2004, PETKM2005, PETKM2006, PETKM2007, PETKM3002, PETKM3003, PETKM7002, PETKM14002, and PETKM22002. Additional exemplary polyester fabrics are catalog numbers P20D, P118, P201, PR150, D117, D1171, D1400, D2000 available from Mohawk Fabrics, Amsterdam, NY 12010.

The fabric can be woven or non-woven, and the fiber size and spacing can be varied to provide a suitable open area for the desired gas permeability while providing enhanced strength to the silicone membrane. Suitable open areas range from about 0.1 to about 3.0 square mm, with fabric fiber size ranging from about 11 to 163 grams per square meter (GSM).

In aspects according to the present disclosure, a fabric suitable for preparing a reinforced membrane 600 may be prepared from natural fibers including cotton and wool. In some aspects, the natural fiber is seed fiber, a leaf fiber, a bast fiber, a skin fiber, a fruit fiber, or a stalk fiber. In other aspects, the natural fiber is hemp, sisal, jute, kenaf, or bamboo. In an aspect, the fabric may be prepared from silk.

In aspects according to the present disclosure, a fabric may be an extruded fabric (also called “extruded netting”). In an aspect, an extruded fabric may be a bi-planar extruded fabric. In another aspect, the extruded fabric may be a mono-planar fabric. Extruded fabric may comprise a netting having a variety of apertures (hole sizes), weights, and thicknesses. Extruded fabrics may be prepared from polypropylene (PP), polyethylene (PE), high density polyethylene (HDPE), medium-density polyethylene (MDPE), low-density polyethylene (LDPE), polypropylene/polyethylene (PP/PE) blends, cross-linked polyethylene (PEX), ultra-high molecular weight polyethylene (UHMWPE).

The reinforcing fabric used to strengthen the silicone membrane can be made from various natural and synthetic materials and fibers, including cotton, silk, wool, polyesters including Dacron®, polyolefins including polyethylene and polypropylene, nylons, polyurethanes, acrylics, cellulose, cellulose acetate, Rayon, polyvinylchloride (PVC) and aramids including Kevlar® Nomex® and Technora®. The fabric can be woven or non-woven, and the fiber size and spacing can be varied to provide a suitable open area for the desired gas permeability while providing enhanced strength to the silicone membrane.

Woven fabrics of the present disclosure may be described by the thread count and have a thread diameter. Woven fabrics comprise warp threads that run lengthwise , and weft or filling threads that run across the width of a fabric at right angles to the warp thread. In woven fabrics comprising monofilaments, equal diameter threads and equal thread counts are present in both the warp and weft directions and square mesh openings (or holes). Monofilament woven fabrics may have different numbers of thread counts in the warp and weft direction resulting in rectangular fabric openings. Woven fabrics are available in a wide variety of thread counts.

In aspects according to the present disclosure, the fabric 620 is between about 50 micrometers (μm) and about 1.5 mm in total thickness. In certain aspects, the maximum thickness of the fabric 750 μm to about 1.0 mm. In an aspect, the thickness of the fabric 620 is between 150 and 300 μm. In an aspect, the thickness of the fabric 620 is between 100 and 450 μm. In an aspect, the thickness of the fabric 620 is between 50 μm and 300 μm. In another aspect, the thickness of the fabric 620 is between 50 μm and 200 μm. In an aspect, the thickness of the fabric 620 is between 200 μm and 300 μm. In an aspect, the thickness of the fabric 620 is about 150 μm. In an aspect, the thickness of the fabric 620 is about 200 μm. In an aspect, the thickness of the fabric 620 is about 250 μm. In an aspect, the thickness of the fabric 620 is about 300 μm.

In the course of developing reinforced membranes 600 of the present disclosure, it is observed that fabrics 620 require a mesh having an open area of more than 75% do not provide a sufficient reinforcement of the silicone to prevent rupture, tearing or puncture, for example when tested in a drop test described in Example 5. Accordingly, the present disclosure provides for and includes, fabrics having a mesh with an open area of between 20% and 60% and a maximal thickness of up to 750 μm. Also included are fabrics having a mesh with an open area of about 55%. In an aspect the mesh opening is about 200 microns and the thread thickness is about 152 microns.

In aspects according to the present disclosure, a fabric 620 may be prepared having a regular, repeating pattern of spaces in the net or network. In other aspects, a fabric 620 of the present disclosure may have an irregular or non-repeating pattern of spaces. In yet another aspect, the fabric 620 may be a random array of open spaces. In another aspect, the fabric 620 may have a honeycomb appearance. In aspects according to the present disclosure, the open spaces within the fabric 620 are round, triangular, square, polygonal, polyhedron, ellipsoid, or spherical.

According to the present disclosure, the fabric 620 comprises a fabric 620 having a percentage of open area of between 40% and 60%. In another aspect, the fabric 620 may have an open area of between 20% and 30%. In an aspect, the fabric 620 may have an open area of between 30% and 40%. In a further aspect, the fabric 620 may have an open area of between 40% and 50%. In yet another aspect, the fabric 620 may have an open area of between 50% and 60%. In certain aspects, the percentage of open area of the fabric 620 may be between 36% and 38%. In an aspect, the percentage of open area is about 37%.

In other aspects, the fabric 620 has a thickness of between 150 μm and 300 μm and has an open area of a fabric 620 between 50% and 70%. In another aspect, the fabric 620 has a thickness of between 150 μm and 300 μm and has an open area of a fabric 620 between 55% and 60%.

Woven monofilament fabrics suitable for the preparation of reinforced silicone membranes 113 of the present disclosure comprise fabric 620 having nominal hole sizes (e.g., mesh openings) ranging from 0.1 to 3 mm².

In aspects according to the present disclosure, suitable fabrics 620 include woven or non-woven fabrics having a pore size of between 0.1 square millimeters (mm²) to about 3.0 mm². As provided herein, fabrics 620 of the present disclosure may have a strand thickness of between 0.15 mm to 0.3 mm. To ensure proper permeability, the fabrics 620 of the present disclosure have an open area of between 50% to 70%. In an aspect, the fabric 620 for reinforcing the silicone membrane is a fabric 620 that has an opening of 1 mm² and a strand thickness of 0.2 mm, and open area of about 55%. In an aspect, the fabric 620 has a strand thickness of 0.0254 millimeters (1 mil). In another aspect, the fabric 620 has a strand thickness of 0.0127 mm (0.5 mil). Suitable fabrics 620 provide for membranes 600 that when incorporated into an inner collapsible blood container 102 that can withstand drop testing from a height of about 6 feet.

In an aspect according to the present disclosure, a collapsible blood container 102 can be manufactured from silicone by various molding methods such as compression molding, injection molding, and insert molding, and also adhesive bonding of silicone sheets using silicone adhesives. In one aspect according to the present disclosure, a pair of silicone sheets are bonded together around the periphery with a section of silicone inlet tubing in place in the seam using silicone adhesive. In another aspect according to the present disclosure, a silicone liquid rubber is injection molded over a form to create a three-sided shape, which is then further bonded to closure on the remaining fourth side around a silicone inlet tube using a silicone adhesive. In another aspect according to the present disclosure, a silicone liquid rubber is injection molded over a form to create a three-sided shape, which is then insert molded onto a closure shape on the remaining fourth side that incorporates an inlet tubing into the closure shape.

The present disclosure provides for, and includes, a collapsible blood container 102 having resistance to tearing. As used herein, “tear resistance” or “tear strength” is measured in kN/m. In aspects according the present disclosure, the collapsible blood container 102 should be prepared from oxygen permeable materials that are also resistant to tearing. Measures of tear resistance are known in the art, for example, ASTM D-412, which can also be used to measure tensile strength, modulus, and elongations. In certain aspects, collapsible blood container 102 should be prepared from oxygen permeable materials that are resistant to the formation of a tear (e.g., tear initiation). Methods of measuring tear initiation and tear propagation are known in the art, for example ASTM D-624. Other methods include measuring the tensile strength and the elongation at break according to DIN 53 504-S1.

In an aspect according to the present disclosure, a collapsible blood container 102 should be prepared from oxygen permeable materials having a tensile strength of at least 2.4 N/mm².

The present disclosure provides for, and includes, sorbents capable of binding to and removing oxygen from an environment. Unless provided otherwise, the term “sorbent” refers to oxygen sorbents and scavengers. As used herein, “oxygen scavenger” or “oxygen sorbent” is a material that binds irreversibly to or combines with O₂ under the conditions of use. The term “oxygen sorbent” may be used interchangeably herein with “oxygen scavenger.” In certain aspects according the present disclosure, a material may bind to or combines with oxygen irreversibly. In other aspects, oxygen may bind to a sorbent material and have a very slow rate of release, k_{off}. In an aspect, the oxygen may chemically react with some component of the material and be converted into another compound. Any material where the off-rate of bound oxygen is much less than the residence time of the blood can serve as an oxygen scavenger. Suitable sorbents as used herein are described in detail in International Application Nos. PCT/US2016/02179 and PCT/US2016/029069.

As used herein, “carbon dioxide scavenger” is a material that binds to or combines with carbon dioxide under the conditions of use. The term “carbon dioxide sorbent” may be used interchangeably herein with “carbon dioxide scavenger.” In certain aspects, carbon dioxide sorbents may be non-reactive, or minimally reactive with oxygen. In other embodiments, oxygen sorbents may exhibit a secondary functionality of carbon dioxide scavenging. Carbon dioxide scavengers include metal oxides and metal hydroxides. Metal oxides react with water to produce metal hydroxides. The metal hydroxide reacts with carbon dioxide to form water and a metal carbonate. In certain aspects according the present disclosure, a material may bind to or combine with CO₂ irreversibly. In aspects according to the present disclosure, a material may bind CO₂ with higher affinity than hemoglobin. In other aspects, a sorbent material may bind CO₂ with high affinity such that the carbonic acid present in the blood or RBC cytoplasm is released and absorbed by the sorbent. In other aspects, CO₂ binds to a sorbent material and has a very slow rate of release, k_{off}. In an aspect, the carbon dioxide can chemically react with some component of the material and be converted into another compound. Suitable carbon dioxide scavengers as used herein are described in detail in U.S. Provisional Application Nos. 62/131,130 and 62/151,957.

The users of the collapsible container require convenient filling and removal of the contents, and must be able to empty the contents within 2 minutes per the ISO 3826standard for blood containers. The outer receptacle can reduce the filling time by constraining the collapsible container and preventing it from expanding. Thus, in some embodiments, the blood storage device is further comprised of an expansion feature to allow for unrestricted filling of the collapsible container. In some embodiments the expansion feature is comprised of a gusseted fold along one or more edges of the outer receptacle. Typically, a fold of about ¼ inch is adequate to provide for expansion of the inner container, and the pleats of the fold are sealed into the seams at the ends. In some embodiments, the expansion feature is comprised of a third panel of barrier film sealed along the bottom of the outer receptacle, providing for a three-dimensional bag.

During the development of the oxygen depletion device 10, it was discovered that the size, shape, and number of chambers of an inner collapsible blood container 102 needed to be controlled in order to obtain suitable depletion kinetics. More particularly, even using highly permeable materials, using standard blood bag configurations proved inadequate and had significantly slower reaction kinetics. Not to be limited by theory, it is hypothesized that deoxygenation is a multistep process including release of dissolved oxygen from hemoglobin, diffusion of the dissolved oxygen within the red blood cell cytoplasm, and diffusion of the dissolved oxygen through the red blood cell membrane. Also not to be limited by theory, it is hypothesized that the high concentration of hemoglobin, having very high affinity for oxygen, greatly decreases the diffusion rate of the dissolved oxygen within the cytoplasm. Similarly, the diffusion of dissolved oxygen once it passes through the plasma membrane to the plasma is further limited by absorption and binding to other red cells. Again, not to be limited by theory, it is hypothesized that an additional diffusion barrier for the dissolved oxygen occurs at the gas permeable membrane where it not only needs to pass through the membrane, but also changes state from the dissolved phase to the gaseous phase. Subsequent diffusion and adsorption by the sorbent occurs in a gaseous state and is maximized by incorporating and maintaining a headspace within the outer receptacle 101. Accordingly, it is believed that the diffusion of the gaseous oxygen is maximized by maintaining the concentration gradient within the headspace from the surface of the inner collapsible blood container 102 to the oxygen sorbent 103. Also not to be limited by theory, it is thought that by selecting sorbents that have high absorption kinetics, high binding capacity, and combinations of both, a suitable diffusion gradient for the gaseous oxygen is maintained to drive the rapid kinetics of oxygen depletion in oxygen depletion device 10.

The present disclosure provides for, and includes, an oxygen depletion device 10 for depleting oxygen from blood that comprises an inner collapsible blood container 102 having a surface to volume ratio of between 0.05 centimeters²/milliliter (cm²/ml) and 5.0 cm²/ml enclosed within an outer receptacle 101. In certain aspects, an oxygen depletion device 10 for depleting oxygen from blood comprises an inner collapsible blood container 102 having a surface to volume ratio of between 0.08 cm²/ml and 4.0 cm²/ml enclosed within an outer receptacle 101 when filled with blood for oxygen depletion. In some aspects, an oxygen depletion device 10 for depleting oxygen from blood comprises an inner collapsible blood container 102 having a surface to volume ratio of between 0.09 cm²/ml and 3.8 cm²/ml enclosed within an outer receptacle 101 when filled with blood for oxygen depletion.

As used herein, surface to volume ratios are defined with respect to a standard unit of whole blood, about 1 pint or 450-500 ml. As is evident to a person of skill in the art, collection of less than a unit of blood results in an even lower surface to volume ratio and the oxygen depletion device 10 is suitable for collecting a fraction of a unit of blood without modification. For the collection of more than a unit of blood, the size of the collapsible blood container 102 would need to be adjusted to provide for the desirable rapid kinetics of blood depletion. Modifications of the sort necessary to adapt an oxygen depletion device 10 for the collection of more than a unit of blood is within the level of ordinary skill in the art.

The present disclosure further includes and provides for oxygen depletion device 10 for the collection and depletion of packed red blood cells. A full unit of packed red blood cells in an additive solution comprises about 280±60 ml.

In an aspect according to the present disclosure, the surface to volume ratio of a collapsible blood container 102 is at least 0.9 centimeters/milliliter (cm²/ml) when filled with blood for oxygen depletion. Not to be limited by theory, it is believed that by increasing the surface to volume ratio, the diffusion limitations imposed by blood itself, particularly by the red blood cells and hemoglobin, can be overcome by decreasing the diffusion distance of the dissolved oxygen within the inner collapsible blood container 102. In an aspect, the surface to volume ratio of a blood container 102 is at least 1.0 cm²/ml when filled with blood for oxygen depletion. In another aspect, the surface to volume ratio of a collapsible blood container 102 is at least 1.5 cm²/ml when filled with blood for oxygen depletion. In a further aspect, the surface to volume ratio of a collapsible blood container 102 is at least 2.0 cm²/ml when filled with blood for oxygen depletion. In some aspects, the surface to volume ratio of a collapsible blood container 102 is at least 3.0 cm²/ml when filled with blood for oxygen depletion. In yet other aspect, the surface to volume ratio of a collapsible blood container 102 is at least 4.0 cm²/ml when filled with blood for oxygen depletion.

The present disclosure also includes and provides for increasing the kinetics of deoxygenation of blood by modifying the dimensions of the inner collapsible blood container 102. Not to be limited by theory, the average diffusion distance of a red blood cell in blood minimized as the height is decreased leading to increased deoxygenation kinetics. In certain aspects according the present disclosure, the collapsible blood container 102 is 12.5 cm by 17.5 cm by 0.002 cm before filling with blood, and about 2.0 cm in height after filling with blood. In other aspects according the present disclosure, the collapsible blood container 102 is 17.5 cm by 28.0 cm by 0.04 cm before filling with blood, and about 2.0 cm in height after filling with blood. In other aspects according the present disclosure, the collapsible blood container 102 is 25.0 cm by 60.0 cm by 0.04 cm before filling with blood, and about 0.3 cm in height after filling with blood.

In certain aspects, the height of a collapsible blood container 102 is no greater than 0.002 cm. In an aspect the height of a collapsible blood container 102 is no greater than 0.04 cm. In certain aspects, the height of a collapsible blood container 102 is between 0.002 and 0.04 cm. When filled with blood, the height of a collapsible blood container 102 is no greater than 0.3 cm. In an aspect the height of a collapsible blood container 102 when filled with blood is no greater than 1.5 cm. In certain aspects, the height of a collapsible blood container 102 when filled with blood is between 0.3 cm and 2.5 cm.

The present disclosure also includes and provides for an oxygen depletion device 10 having dimensions suitable for incorporation of existing blood collection protocols using existing equipment. Design of an oxygen depletion device 10 with recognition to existing technologies reduces capital costs in centralized processing centers and further provides for increased consistency and reliability. As used herein, the dimensions of an oxygen depletion device 10 is primarily limited to the length and width of the outer receptacle 101 where the height of the bag is determined by the requirements of the collapsible blood container 102 to contain about a pint or 450 to 500 ml. of blood, which is equivalent to a “unit of blood”. The height of an oxygen depletion device 10 is further constrained by the presence of one or more sorbent packets and devices included to maintain an appropriate headspace. In view of these considerations, it become apparent that constraints on the dimension of the outer receptacle 101 of an oxygen depletion device 10 necessarily limits the dimensions of a collapsible blood container 102. Accordingly, a collapsible blood container 102 may be divided into one or more chambers in fluid communication with each other.

In aspects according to the present disclosure, an oxygen depletion device 10 is designed to be incorporated into existing blood agitation equipment. In certain aspects, an oxygen depletion device 10 is dimensioned to efficiently utilize the space available in agitator and mixing tables. In an aspect, an oxygen depletion device 10 is dimensioned to maximally utilize the area available in a platelet agitator, for example a Helmer Labs Platelet Agitator, Model PF96. Suitable dimensions of an oxygen depletion device 10 include those that allow for 1, 2, 4, 6, 8, 10 or more bags to be placed on a flat agitator or mixer surface.

In an aspect, the area of an oxygen depletion device 10 lying flat is between 150 and 250 cm². In another aspect an oxygen depletion device 10 lying flat is between 450 and 550 cm². In another aspect an oxygen depletion device 10 lying flat is between 1400 and 1500 cm². In another aspect an oxygen depletion device 10 lying flat is between 150 and 1500 cm².

As is evident, an oxygen depletion device 10 having a defined size necessarily constrains the dimensions of a collapsible blood container 102 according to the present disclosure. In certain aspects, a collapsible blood container 102 is further limited by a specified surface to volume ratio. In accordance with these limitations, the present disclosure provides for, and includes, a collapsible blood container 102 having two or more chambers in fluid communication with each other.

The oxygen depletion container device can be constructed in such a manner that allows for the blood volume to area of bag to be optimized against the overall size of the oxygen depletion container device, while exposing more of the blood volume to the material with oxygen permeability in the utilized space. The blood volume can be contained in a collapsible blood container 102 having two or more chambers that allow for their specific arrangement within the outer receptacle 101. In certain aspects, the oxygen depletion device 10 height, when placed onto a surface, does not occupy impractical space in the intended mixing apparatus. The chambers can be arranged side to side, stacked on top of one another, partially stacked onto each other, staggered in a row, or saddled on top of each other onto one or more stacking heights. Sorbent 103 can be positioned over or between chambers as needed. Chambers may be filled and drained individually or in unison when such chambers are connected via tubing or fluid conduits that allow for easy filling and draining. It would be understood that the arrangement and interconnection of collapsible blood containers 102 having two or more chambers can be performed by a person of skill in the art.

In certain aspects, a collapsible blood container 102 comprises two or more chambers. In an aspect, a collapsible blood container 102 can have two chambers placed side by side or end to end depending on the dimensions. In another aspect, a collapsible blood container 102 can have three chambers placed side by side or end to end depending on the dimensions. In yet another aspect, a collapsible blood container 102 can have three chambers placed side by side or end to end depending on the dimensions. A person of ordinary skill could prepare additional configurations of a collapsible blood container 102 having multiple chambers placed in adjacent positions and orientations to maximize the utilization of space.

In other aspects provided for and included in the present disclosure, a collapsible blood container 102 may comprise two or more chambers that are stacked. When in a stacked configuration, to maintain optimal gas diffusion rates, spacers or meshes are included to ensure the separation of adjacent chambers. In certain aspects, the space between a stacked chamber further includes one or more sorbent sachets in order to maintain optimal gas diffusion rates. In certain aspects, two chambers may be stacked. In another aspect, three chambers may be stacked. In yet another aspect, four chambers may be stacked.

The present disclosure provides for, and includes, a collapsible blood container 102 comprising a combination of stacked and adjacent chambers. As provided herein, the number and stacking of chambers of a collapsible blood container 102 further comprises a surface to volume ratio of the combined chambers of at least 0.4 cm²/ml. Additional variations consistent with the present disclosure can be prepared by one of ordinary skill in the art.

The present disclosure provides for, and includes, an oxygen depletion device 10 for depleting oxygen from blood comprising an outer receptacle 101 substantially impermeable to oxygen, inner collapsible blood container 102 that is permeable to oxygen and an oxygen sorbent situated within said outer receptacle wherein the collapsible blood container 102 further comprises one or more mixing structures that increase mixing of the blood during oxygen depletion. In certain aspects, the mixing structures are incorporated into the structure of the collapsible blood container 102. In other aspects, the mixing structures are added to the inside of, but not physically joined to the collapsible blood container 102. In yet other aspects, a mixing structure is a structure outside of the collapsible blood container 102 that restricts or modifies the shape of the container 102 to decrease or disrupt laminar flow. Mixing structures according to the present disclosure are designed to increase blood movement in the collapsible blood container 102, increase turbulent flow within the collapsible blood container 102, or combinations of both. Importantly, mixing structures and mixing should not significantly increase lysis, or damage to, the red blood cells.

In aspects according to the present disclosure, a mixing structure is included in the structure of membrane 113. In certain aspects, a mixing structure in membrane 113 comprises ridges, bumps, or protrusions on the inside of the collapsible blood container 102 and are in contact with the blood. As provided herein, such mixing structures can include the fabric reinforced surface of a reinforced membrane 600 or the features 701 of a reinforced membrane 700 when the surfaces are incorporated on the inside surface of a collapsible blood container 102. In certain aspects, the reinforcing features 701 may be present on both sides of a silicone membrane 113 of a reinforced membrane 700 wherein the features 701 provide both reinforcing functions and mixing functions. In an aspect, a mixing structure in membrane 113 comprises one or more ridges. In an aspect, the one or more ridges extend across the full width or length of the inner surface of collapsible blood container 102. In other aspects, the ridges alternate and may be staggered. In certain aspects, the mixing structure in membrane 113 comprises bumps or other protrusions designed to disrupt laminar flow and induce turbulence. Similarly, in certain aspects, the mixing structure in membrane 113 comprises depressions designed to disrupt laminar flow and induce turbulence. In certain aspects, the mixing structures are baffles incorporated into membrane 113. Baffles are flow directing vanes or panels. In some aspects, a mixing structure comprising one or more baffles may be incorporated into a second membrane 114.

In certain aspects, a mixing structure is contained within the collapsible blood container 102. In an aspect, a mixing structure within the collapsible blood container 102 comprises one or more beads or balls that aid in mixing when the collapsible blood container 102 is agitated. In another aspect, a mixing structure within the collapsible blood container 102 comprises one or more strings or elongated structures that aid in mixing when the collapsible blood container 102 is agitated. In yet another aspect, a mixing structure within the collapsible blood container 102 comprises a mesh or aids in mixing when the collapsible blood container 102 is agitated.

The present disclosure provides for, and includes, an oxygen depletion device having an outer receptacle 101 that is substantially impermeable to oxygen enclosing an inner collapsible blood container 102 and providing a headspace. In an aspect, the oxygen sorbent 103 is disposed within the headspace thereby creating and an oxygen depleted state within the headspace. In an aspect, said oxygen sorbent 103 disposed in the headspace further maintains the headspace in an oxygen depleted state by removing oxygen that may enter through the outer receptacle 101 or through the one or more inlets/outlets 30.

Mainaining the headspace in an oxygen depleted state provides for improved shelf life for oxygen depletion device 10. In an aspect, the shelf life of an assembled oxygen depletion device 10 has a shelf life of at least 24 months. In another aspect, the oxygen depletion device 10 has a shelf life of at least 12 months after assembly of the components. In an aspect according to the present disclosure, the assembled oxygen depletion device 10 meets ISTA-2A standards.

In certain aspects of the present disclosure, the headspace provides for improved processing times. For oxygen depletion device 10, removing ambient air present or inert flushing gas from the assembly prior to sealing the outer receptacle 101 reduces the volume of the headspace. Applying a vacuum to the outer receptacle 101 prior to sealing reduces the volume of the headspace and decreases the total volume of the assembled oxygen depletion device. While reduced overall headspace volume provides for reduced shipping volume, it can result in increased filling times by constraining the collapsible blood container 102. In certain aspects, the headspace may be flushed with nitrogen gas and then sealed under slightly less than ambient pressure to provide a reduced headspace volume in the oxygen depletion device 10 without significantly increasing the fill and process time.

In certain aspects, the headspace may be initially depleted of oxygen by flushing the headspace with nitrogen. In an aspect, the headspace of oxygen depletion device 10 is flushed with nitrogen gas prior to sealing the outer receptacle 101. In an aspect, the flushing gas is ≥99.9% nitrogen gas.

The present disclosure includes and provides for oxygen depletion device 10 having inner collapsible blood container 102 divided into two or more compartments. In certain aspects, an oxygen depletion device 10, having a collapsible blood container 102 divided into multiple compartments has a headspace of between 10 and 500 ml per compartment. In an aspect the headspace is between 20 and 400 ml per compartment. In another aspect the headspace volume is between 60 and 300 ml per compartment. In a further aspect, the headspace volume is between 100 and 200 ml per compartment of a collapsible blood container. In an aspect, an oxygen depletion device 10 having inner collapsible blood container 102 divided into compartments has a headspace of about 10 ml per compartment. In another aspect, the headspace is about 100 ml to about 200 ml per compartment. In another aspect the headspace is about 300 ml to about 500 ml per compartment.

The present disclosure includes and provides for oxygen depletion device 10 having inner collapsible blood container 102 divided into two or more compartments. In certain aspects, an oxygen depletion device 10, having a collapsible blood container 102 divided into two compartments has a headspace of between 20 and 1000 ml. In an aspect the headspace is between 100 and 800 ml. In another aspect the headspace volume is between 200 and 700 ml. In a further aspect, the headspace volume is between 300 and 500 ml for a two compartment collapsible blood container. In an aspect, an oxygen depletion device 10 having inner collapsible blood container 102 divided into two compartments has a headspace of about 700 ml. In another aspect, the headspace is about 200 ml to about 700 ml. In another aspect the headspace is about 300 ml to about 500 ml.

The present disclosure includes and provides for oxygen depletion device 10 having inner collapsible blood container 102 divided into two or more compartments. In certain aspects, an oxygen depletion device 10, having a collapsible blood container 102 divided into three compartments has a headspace of between 20 and 1000 ml. In an aspect the headspace is between 100 and 800 ml. In another aspect the headspace volume is between 200 and 700 ml. In a further aspect, the headspace volume is between 400 and 600 ml for a three compartment collapsible blood container. In an aspect, an oxygen depletion device 10 having inner collapsible blood container 102 divided into three compartments has a headspace of about 800 ml. In another aspect, the headspace is about 200 ml to about 700 ml. In another aspect the headspace is about 400 ml to about 600 ml.

The present disclosure includes and provides for an oxygen depletion device 10 having an inner collapsible blood container 102 and further including one or more spacers 111 that ensure the separation of the outer receptacle 101 and the inner collapsible blood container 102. The spacer 111 provides for the maintenance of the headspace in the oxygen depletion device to ensure efficient diffusion of the oxygen from the surface of membrane 113 to the sorbent 103. A spacer 111 can be prepared from one or more of the materials selected from the group consisting of a mesh, a molded mat, a woven mat, a non-woven mat, a strand veil, and a strand mat. In certain aspects, the spacer 111 can be integrated directly into the collapsible blood container 102 as ribs, dimples, or other raised feature that maintains a separation between the outer receptacle 101 and the inner collapsible blood container 102. The present specification also includes and provides for a spacer 111 to be integrated into the outer receptacle 101 as ribs, dimples, or other suitable raised feature capable of maintaining a separation between the outer receptacle 101 and the inner collapsible blood container 102.

The present disclosure also includes and provides for inner collapsible blood containers 102 that further comprise a window 112. As used herein, a window 112 is made of a transparent material and is bonded or otherwise incorporated into the inner collapsible blood container 102. In accordance with the present disclosure, suitable materials for window 112 are blood compatible. In certain aspects, materials suitable for a window 112 are oxygen impermeable. In other aspects, materials suitable for a window 112 are oxygen impermeable. The size of a window 112 need only be large enough to provide observation of the blood.

Also included and provided for by the present disclosure are collapsible blood containers having bis(2-ethylhexyl) phthalate (DEHP). DEHP is included in most PVC based blood storage bags as a plasticizer where it has been observed that DEHP provides a protective effect to stored red blood cells. See U.S. Pat. No. 4,386,069 issued to Estep. In certain aspects, an oxygen depletion device 10 may further include DEHP incorporated in the inner collapsible blood container 102. In other aspects, DEHP may be provided separately within the inner collapsible blood container 102.

The present disclosure provides for, and includes, an oxygen depletion device 10 that does not include DEHP. It has been hypothesized that DEHP may act as an endocrine disruptor and certain regulatory agencies are considering ordering the removal of DEHP from blood bags. It has been observed that DEHP may not be necessary when red blood cells are stored anaerobically. See, International Patent Publication No. WO 2014/134503, hereby incorporated by reference in its entirety. Accordingly, in certain aspects, oxygen depletion device 10 entirely excludes DEHP from all blood contacting surfaces. In other aspects, oxygen depletion device 10 limits DEHP containing surfaces to tubing, ports, and inlets such as those illustrated in the Figures at, for example, 106 and 205. In an aspect, oxygen depletion device 10 excludes a DEHP containing collapsible blood container 102.

The present disclosure also includes and provides for inner collapsible blood containers 102 that include an inlet/outlet 130. As provided below, an inlet/outlet 130 may be incorporated into the inner collapsible blood containers 102 during assembly using a frame 120 or may be integrated into the inner collapsible blood container 102 during manufacture using compression or blow molding. An inlet/outlet 130 may comprise silicone. In other aspects, inlet/outlet 130 may comprise materials selected from the group consisting of ethylene-vinyl acetate (EVA), poly(ethylene-vinyl) acetate (PEVA), polypropylene (PP), polyurethane (PU), polyester (PES), polyethylene terephthalate (PET), polyethylene (PE), high-density polyethylene (HDPE), polyvinyl chloride (PVC), polyvinylidene chloride (PVDC), low-density polyethylene (LDPE), polypropylene (PP), polystyrene (PS), high impact polystyrene (HIPS), polyamides (PA) (e.g., nylon), acrylonitrile butadiene styrene (ABS), polycarbonate (PC), polycarbonate/acrylonitrile butadiene styrene (PC/ABS), polyurethanes (PU), melamine formaldehyde (MF), plastarch material, phenolics (PF), polyetheretherketone (PEEK), polyetherimide (PEI) (Ultem), polylactic acid (PLA), polymethyl methacrylate (PMMA), polytetrafluoroethylene (PTFE), urea-formaldehyde, ethylene vinyl alcohol copolymer (EVOH), and polyamide.

As used herein, the term “blood” refers to whole blood, leukoreduced RBCs, platelet reduced RBCs, and leukocyte and platelet reduced RBCs. The term blood further includes packed red blood cells, platelet reduced packed red blood cells, leukocyte reduced packed red blood cells (LRpRBC), and leukocyte and platelet reduced packed red blood cells. The temperature of blood can vary depending on the stage of the collection process, starting at the normal body temperature of 37° C. at the time and point of collection, but decreasing rapidly to about 30° C. as soon as the blood leaves the patient's body and further thereafter to room temperature in about 6 hours when untreated, and ultimately being refrigerated at between about 4° C. and 6° C.

As used herein, the term “whole blood” refers to a suspension of blood cells that contains red blood cells (RBCs), white blood cells (WBCs), platelets suspended in plasma, and includes electrolytes, hormones, vitamins, antibodies, etc. In whole blood, white blood cells are normally present in the range between 4.5 and 11.0×10⁹ cells/L and the normal RBC range at sea level is 4.6-6.2×10¹²/L for men and 4.2-5.4×10¹²/L for women. The normal hematocrit, or percent packed cell volume, is about 40-54% for men and about 38-47% for women. The platelet count is normally 150-450×10⁹/L for both men and women. Whole blood is collected from a blood donor, and is usually combined with an anticoagulant. Whole blood, when collected is initially at about 37° C. and rapidly cools to about 30° C. during and shortly after collection, but slowly cools to ambient temperature over about 6 hours. Whole blood may be processed according to methods of the present disclosure at collection, beginning at 30-37° C., or at room temperature (typically about 25° C.). As used herein, a “unit” of blood is about 450-500 ml including anticoagulant.

As used herein, “red blood cells” (RBCs) includes RBCs present in whole blood, leukoreduced RBCs, platelet reduced RBCs, and leukocyte and platelet reduced RBCs. Human red blood cells in vivo are in a dynamic state. The red blood cells contain hemoglobin, the iron-containing protein that carries oxygen throughout the body and gives red blood its color. The percentage of blood volume composed of red blood cells is called the hematocrit. As used herein, unless otherwise limited, RBCs also includes packed red blood cells (pRBCs). Packed red blood cells are prepared from whole blood using centrifugation techniques commonly known in the art. As used herein, unless otherwise indicated, the hematocrit of pRBCs is about 50%.

The present disclosure provides for, and includes, a blood storage device 20, for storing oxygen depleted blood and maintaining the blood in a deoxygenated state during the storage period. Certain anaerobic blood storage devices (ASB) are known in the art, including for example U.S. Pat. No. 6,162,396 to Bitensky et al. The anaerobic blood storage devices of the prior art did not include ports and inlets designed to be substantially impermeable to oxygen. Accordingly, the prior art anaerobic storage devices had poor shelf lives prior to use and were susceptible to significant ingress of oxygen. As provided in the present disclosure, an improved blood storage device 20 comprising features directed to maintaining the integrity of the device while allowing for the sampling of the blood that occurs during storage and blood banking. The improved ASB also provides for improved diffusion of oxygen from the blood, providing for additional depletion during the storage period.

The blood storage device 20 comprises an outer receptacle 201 that is substantially impermeable to oxygen, a collapsible blood container 202 comprising a locating feature 203 adapted to align the collapsible blood container 202 within the geometry of the outer receptacle 201; at least one inlet/outlet 30 comprising connecting to the collapsible blood container 202 and a bond 206 to the outer receptacle 201, wherein the bond 206 to the outer receptacle 201 is substantially impermeable to oxygen and an oxygen sorbent 207 situated within the outer receptacle 201.

As used herein, an outer receptacle 201 is at least equivalent to an outer receptacle 101. Also as used herein, an inner collapsible blood container 202 includes blood containers as provided above for an inner collapsible blood container 102 but also provides for collapsible blood containers 202 comprising materials that are less permeable to oxygen, such as PVC. Also as provided herein, oxygen sorbent 207 is at least equivalent to sorbent 103 and may be provided in sachets as discussed above.

Like a reinforced membrane 600, a reinforced membrane 700 comprises a silicone membrane 113 that is substantially permeable to oxygen and reinforced with a features 701. Reinforced membranes 700 are suitable for the preparation of inner collapsible containers 102 for use in oxygen depletion devices 10. In certain aspects, the reinforced silicone membranes 700 may be further characterized as having a relatively smooth surface for contact with the blood or blood component for depletion. In other aspects, the blood contacting surface can be modified with additional features to provide for additional mixing. In certain aspects, the features 701 of a membrane 700 can be presented for contact with the blood in an inner collapsible container 102 to improve mixing.

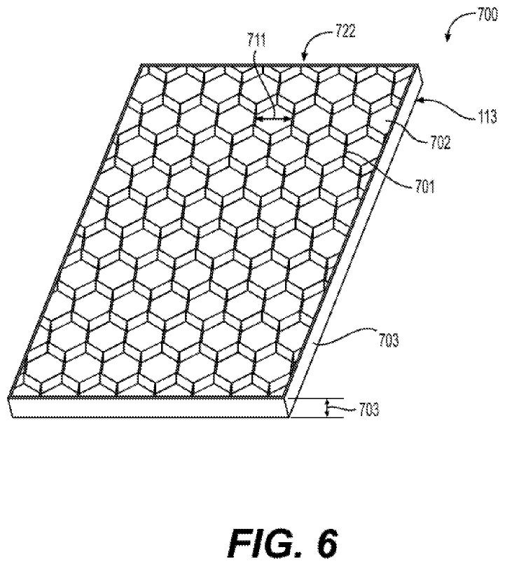

The present disclosure provides for, and includes a membrane 700 that is illustrated generally by way of example, but not by way of limitation, as shown in FIG. 6. More specifically, FIG. 6 presents an aspect of a membrane 700 having raised features 701 arranged in a pattern 722. As will be provided below, the arrangement of raised features 701 can be formed in a variety of patterns 720. Referring to FIG. 6, the general aspects of a reinforced silicone membrane 700 comprises a silicone membrane 113 having an area 702 having an average thickness 703 of less than 100×10⁻⁶ M (μm), and features 701. Like reinforced membranes 600, reinforced membranes 700 provide for the manufacture of inner collapsible blood containers 102 that may help comply with ISO standard 3826-1:2013 that requires that plastic collapsible blood containers shall not show leakage when placed between two plates and subjected to an internal pressure of 50 kPa above atmospheric pressure for 10 minutes. Reinforced membranes 700 overcome the strength limitations of silicone membranes 113 when the thickness is reduced to less than 100 μm. As provided herein, reinforced membranes 700 provide for a silicone membrane 113 has a thickness 703 of between 5 and 100 μm.

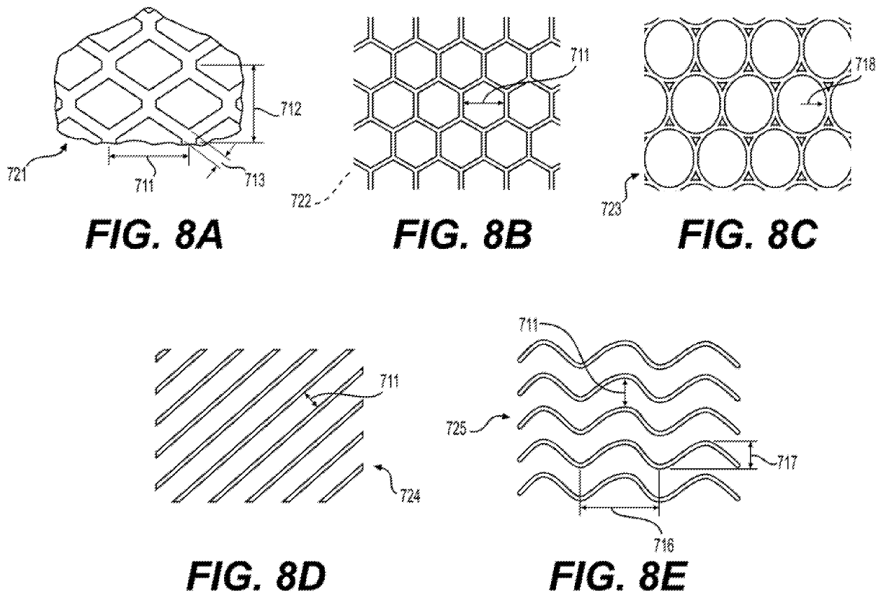

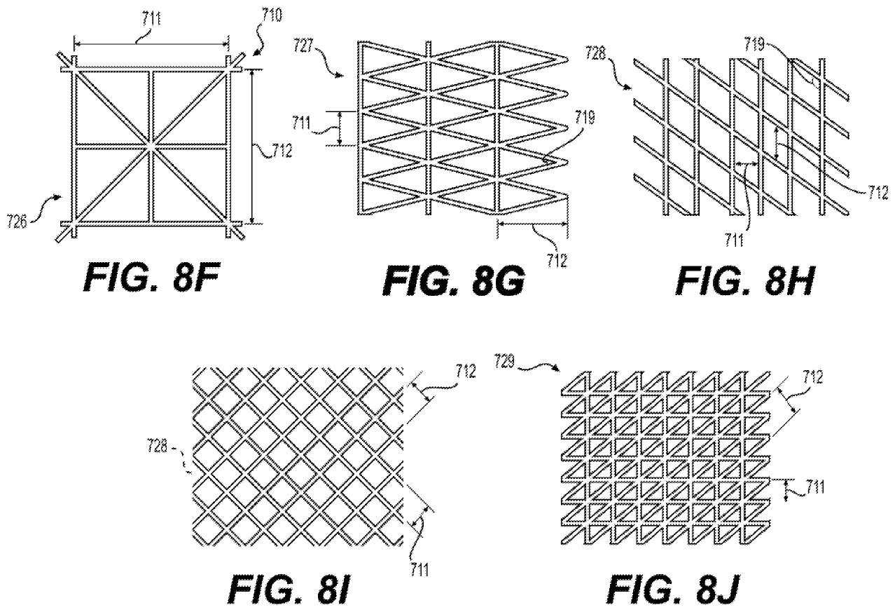

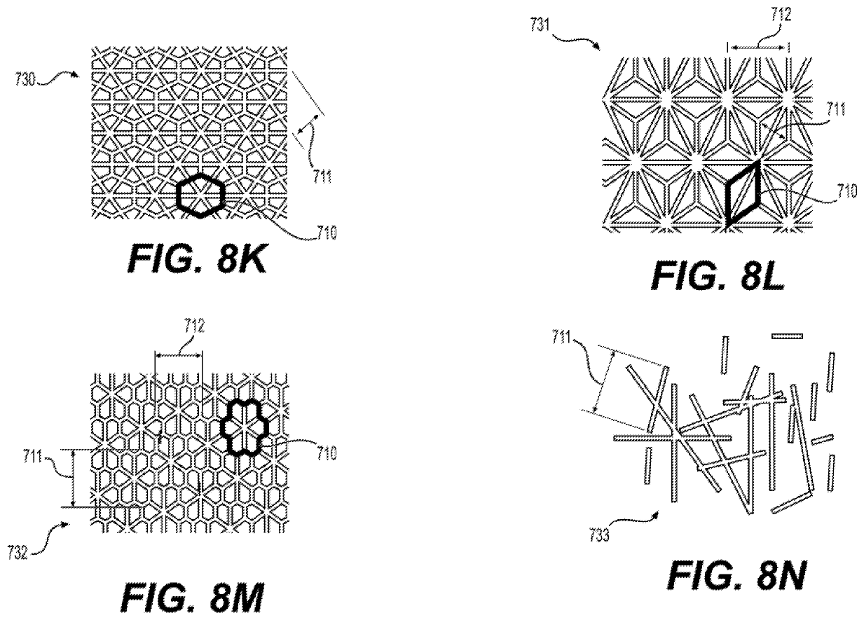

Referring to FIG. 6, the raised features 701 are arranged in a pattern 722 (hexagonal) that encloses an area 702. As used herein, area 702 refers to the areas of silicone membrane 113 that are less than 100 μm in average thickness (e.g., thickness 703). While shown in FIG. 6 as being surrounded by features 701, in aspects according to the present specification, area 702 does not need to be surrounded by raised features. As will be discussed below, the raised features 701 may be arranged as raised lines (e.g., FIG. 8D, pattern 724), waved lines (e.g., FIG. 8E, pattern 724), or random features (e.g., FIG. 8N, pattern 733). Accordingly, area 702 refers to those areas of membrane 700 that are less than 100 μm and are not areas having features 701.

In aspects of the present disclosure, silicone membrane 113 has an average thickness 703 of less than 100×10⁻⁶ M (μm) in areas 702. As used herein, the thickness 703 refers to the average thickness of area 702. In aspects of the present disclosure, the average thickness 703 of area 702 can vary ±10%. In certain aspects, silicone membrane 113 has an average thickness 703 of 50 μm. In some aspects, silicone membrane 113 has an average thickness 703 of 40 μm. In another aspect, silicone membrane 113 has an average thickness 703 of 30 μm. In certain aspects, silicone membrane 113 has an average thickness 703 of 25 μm. In other aspects, silicone membrane 113 has an average thickness 703 of 20 μm. In certain aspects, silicone membrane 113 has an average thickness 703 of less than 50 μm. In some aspects, silicone membrane 113 has an average thickness 703 of less than 40 μm. In other aspects, silicone membrane 113 has an average thickness 703 of less than 30 μm. In yet other aspects, silicone membrane 113 has an average thickness 703 of less than 20 μm. In certain aspects, silicone membrane 113 has an average thickness 703 of between 5 μm and 95 μm. In other aspects, silicone membrane 113 has an average thickness 703 of between 20 μm and 95 μm. In other aspects, silicone membrane 113 has an average thickness 703 of between 5 μm and 50 μm. In a further aspect, silicone membrane 113 has an average thickness 703 of between 5 μm and 20 μm. In yet other aspects, silicone membrane 113 has an average thickness 703 of between 10 μm and 40 μm. In certain aspects, silicone membrane 113 has an average thickness 703 of between 10 μm and 30 μm. In yet other aspects, silicone membrane 113 has an average thickness 703 of between 10 μm and 25 μm. In a further aspect, silicone membrane 113 has an average thickness 703 of between 15 μm and 35 μm.

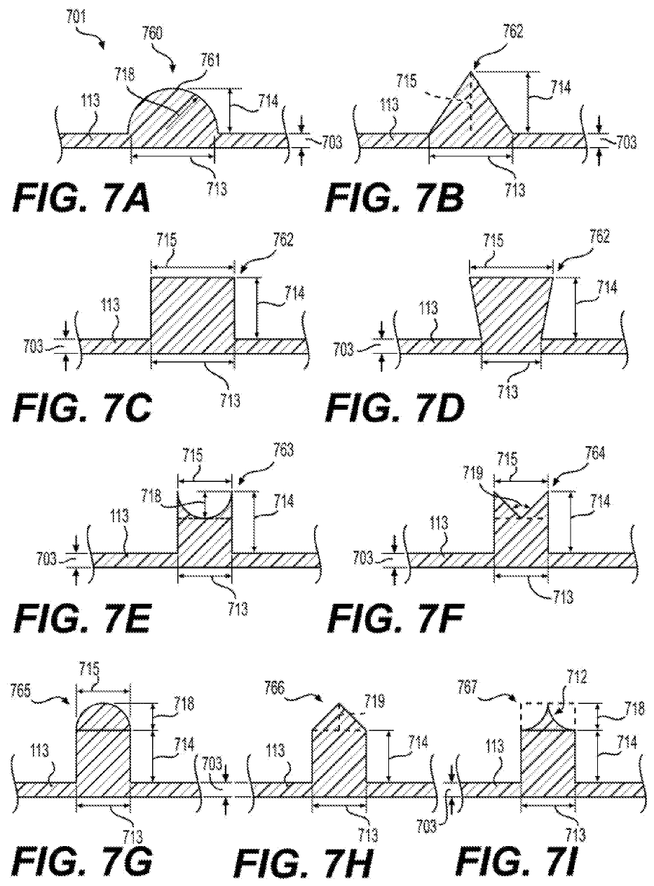

The present disclosure provides for and includes, a reinforced membrane 700, as illustrated in FIG. 6, having features 701 that reinforce the silicone membrane 113. Features 701 provide for mechanical support and strengthen the membrane to reduce tearing splitting, or rupturing when the reinforced membrane 700 is used to prepare a collapsible blood container 102. More specifically, the features 701 provide for reinforcement of the silicone membrane 113 and allow for improved structural integrity when devices made with the reinforced membrane 700 are subjected to conditions routinely found, for example, in a blood collection center. These conditions include, for example, stacking, mixing, or centrifuging of filled collapsible blood containers 102. Importantly, the features 701 of a reinforced membrane 700 provide for improved durability and allow for collapsible blood containers 102 prepared therefrom to survive drop tests. As shown in FIG. 7, feature 701 can be prepared having a variety of cross sections 760. As shown in FIG. 8, the features 701 having cross sections 760 can be applied to, or incorporated in, silicone membrane 113 in a variety of patterns 720. Alternatively, as shown in FIG. 8, the features 701 having cross sections 760 can be applied to, or incorporated in, silicone membrane 113 randomly as shown for example in FIG. 8N, pattern 733.

In aspects of the present disclosure, the features 701 may be disposed on one side of membrane 113 or on both sides of silicone membrane 113. In an aspect of the present disclosure, features 701 are disposed on one side of silicone membrane 113. In another aspect of the present disclosure, features 701 are disposed on both sides of the silicone membrane 113. As provided herein, features 701 disposed on one side of silicone membrane 113 may be different than features 701 disposed on the opposite side of silicone membrane 113.

As will be appreciated, less than the entire surface of silicone membrane 113 is covered by features 701. More specifically, in order to retain the desirable oxygen permeability of a reinforced membrane 700, the area 702 having a thickness 703 of less than 100 μm should be maximized. Similarly, the cross section 760 and the pattern 720 are selected to, among other criteria, maximize the strength of the reinforced membrane 700. In other aspects, the cross section 760 and the pattern 720 may be selected to improve mixing of blood in a collapsible blood container 102. In yet other aspects, the cross section 760 and the pattern 720 may be selected to improve diffusion of oxygen in the headspace of an oxygen depletion device 10 whereby the features 701 function as a spacer 110, or a spacer 213 of a blood storage device 20.

The present disclosure provides for, and includes, a reinforced membrane 700 having features 701 covering a percentage of less than 50% of the area of at least one side of silicone membrane 113. That is, features 701, having a width of length 713 and a height of length 714 and cover an area of silicone membrane 113 of less than 50% such that 50% of silicone membrane 113 comprises area 702 having an average thickness 703 of less than 100 μm. The present disclosure provides for, and includes, a reinforced membrane 700 having features 701 covering a percentage of less than 30% of the area of at least one side of silicone membrane 113. That is, features 701, having a width of length 713 and a height of length 714 and cover an area of silicone membrane 113 of less than 50% such that 50% of silicone membrane 113 comprises area 702 having an average thickness 703 of less than 100 μm. In an aspect, features 701 covers between 30 and 50% of the area of at least one side of silicone membrane 113. In an aspect, features 701 covers between 10 and 50% of the area of at least one side of silicone membrane 113. In an aspect, features 701 covers between 10 and 30% of the area of at least one side of silicone membrane 113. In an aspect, features 701 covers between 20 and 40% of the area of at least one side of silicone membrane 113. In another aspect, features 701 covers between 0.5 and 10% of the area of at least one side of silicone membrane 113. In another aspect, features 701 covers between 5.0 and 10% of the area of at least one side of silicone membrane 113. In another aspect, features 701 covers between 10 and 25% of the area of at least one side of silicone membrane 113. In a further aspect, feature 701 covers at least 1% of the area of at least one side of silicone membrane 113. In a further aspect, feature 701 covers at least 5% of the area of at least one side of silicone membrane 113. As provided herein, features 701, covering a percentage of less than 30% of the area of silicone membrane 113 are arranged in a pattern 720. In aspects of the present disclosure, features 701, covering a percentage of less than 30% of the area of silicone membrane 113 are arranged in a pattern selected from the group consisting of pattern 721, pattern 722, pattern 723, pattern 724, pattern 725, pattern 726, pattern 727, pattern 728, pattern 729, pattern 730, pattern 731, pattern 732, and pattern 733, as illustrated in FIG. 8. As provided herein, features 701, covering a percentage of less than 50% of the area of silicone membrane 113 are arranged in a pattern 720. In aspects of the present disclosure, features 701, covering a percentage of less than 50% of the area of silicone membrane 113 are arranged in a pattern selected from the group consisting of pattern 721, pattern 722, pattern 723, pattern 724, pattern 725, pattern 726, pattern 727, pattern 728, pattern 729, pattern 730, pattern 731, pattern 732, and pattern 733, as illustrated in FIG. 8.

The present disclosure provides for and includes, a reinforced membrane 700, having silicone membrane 113 with an average thickness 703 of less than 100×10⁻⁶ M (μm) in area 702, reinforced with features 701 with a cross-section 760, as illustrated in FIG. 7. In aspects of the present disclosure, features 701 with cross-section 760 can have a pattern 720 selected from the group consisting of patterns 721 to 732, and 733, as illustrated in FIG. 8. The present disclosure provides for features 701 having a cross-section 760 selected from the group consisting of 761, 762, 763, 764, 765, 766, and 767, as illustrated in FIG. 7. As provided herein, features 701 have a cross-section 760 having a length 713 perpendicular to length 714. In certain aspects, features 701 have cross-section 760 further comprise a length 715 perpendicular to a length 714. In other aspects, features 701 have cross-section 760 having a length 713 perpendicular to length 714 and further comprise a radius of length 718. In a further aspect, features 701 have cross-section 760 having a length 713 perpendicular to length 714, and further comprise a length 715 perpendicular to a length 714 and a radius of length 718. In another aspect, features 701 have cross-section 760 having a length 713 perpendicular to length 714, a length 715 perpendicular to a length 714, and an angle 719.

The present disclosure provides for and includes, a reinforced membrane 700, having silicone membrane 113 with an average thickness 703 of less than 100×10⁻⁶ M (μm) in areas 702, reinforced with features 701 with a cross-section 760, as illustrated in FIG. 7. In aspects of the present disclosure, features 701 with cross-section 760 can have a pattern 720 selected from the group consisting of patterns 721 to 732, and 733, as illustrated in FIG. 8. The present disclosure provides for features 701 having a cross-section 760 selected from the group consisting of 761, 762, 763, 764, 765, 766, and 767, as illustrated in FIG. 7. As provided herein, features 701 have cross-section 760 having a length 713 of less than 2500 μm, perpendicular to length 714 of less than 2500 μm. In certain aspects, features 701 have cross-section 760 further comprise a length 715 of less than 2500 μm, perpendicular to a length 714 of less than 2500 μm. In other aspects, features 701 have cross-section 760 having a length 713 of less than 2500 μm, perpendicular to length 714 of less than 2500 μm, and further comprise a radius of length 718 of less than 1250 μm. In a further aspect, features 701 have cross-section 760 having a length 713 of less than 2500 μm, perpendicular to length 714 of less than 2500 μm, and further comprise a length 715 of less than 2500 μm, perpendicular to a length 714 and a radius of length 718 of less than 1250 μm. In another aspect, features 701 have cross-section 760 having a length 713 of less than 2500 μm, perpendicular to length 714 of less than 2500 μm, a length 715 of less than 2500 μm, perpendicular to a length 714 of less than 2500 μm, and an angle 719 of less than 60°.

In an aspect of the present disclosure, reinforced membrane 700, has a silicone membrane 113 having a thickness 703 of less than 100 μm and features 701 having a cross-section 761, with a length 714 of less than 2500 μm, and a radius of length 718 of less than 1250 μm. In certain aspects, features 701 having a cross-section 761, with a length 714 of less than 2500 μm, and a radius of length 718 of less than 100 μm. In certain aspects, features 701 have a cross-section 761 having a length 714 of between 250 μm and 1000 μm, and a radius of length 718 of between 125 μm and 500 μm. In other aspects, features 701 have a cross-section 761 having a length 714 of between 100 μm and 500 μm, and a radius of length 718 of between 50 μm and 250 μm. In certain aspects, features 701 have a cross-section 761 having a length 714 of between 100 μm and 2500 μm, and a radius of length 718 of between 50 μm and 1250 μm. In other aspects, features 701 have a cross-section 761 having a length 714 greater than a radius of length 718. In yet other aspects, features 701 have a cross-section 761 having a length 714 less than a radius of length 718. In certain aspects, features 701 have a cross-section 761 having a length 714 of between 100 and 2500 μm. In certain aspects, features 701 have a cross-section 761 having a radius of length 718 between 5 μm and 100 μm.

In an aspect of the present disclosure, reinforced membrane 700, has a silicone membrane 113 having a thickness 703 of less than 100 μm and features 701 having a cross-section 762, with a length 713 of less than 2500 μm, a length 714 of less than 2500 μm and a length 715 of less than 2500 μm. In certain aspects, features 701 have a cross-section 762, with a length 713 of less than 1000 μm, a length 714 of less than 1000 μm and a length 715 of less than 1000 μm. In certain aspects, features 701 have a cross-section 762, with a length 713 of less than 500 μm, a length 714 of less than 500 μm and a length 715 of less than 500 μm. In other aspects, features 701 have a cross-section 762, with a length 713 of between 100 μm and 2500 μm, a length 714 of between 100 μm and 2500 μm, and a length 715 of between 100 μm and 2500 μm. In other aspects, features 701 have a cross-section 762, with a length 713 of between 250 μm and 1000 μm, a length 714 of between 250 μm and 1000 μm, and a length 715 of between 250 μm and 1000 μm. In yet other aspects, features 701 have a cross-section 762, with a length 713 of between 250 μm and 1000 μm, a length 714 of between 250 μm and 1000 μm, and a length 715 equal to length 713. In a further aspect, features 701 have a cross-section 762, with a length 713 of between 250 μm and 1000 μm, a length 714 of between 250 μm and 1000 μm, and a length 715 less than length 713. In a further aspect, features 701 have a cross-section 762, with a length 713 of between 250 μm and 1000 μm, a length 714 of between 250 μm and 1000 μm, and a length 715 greater than length 713. In a further aspect, features 701 have a cross-section 762, with a length 713 of between 250 μm and 1000 μm, a length 714 of between 250 μm and 1000 μm, and a length 715 equal to zero. In aspects of the present disclosure, reinforced membrane 700, has a silicone membrane 113 having a thickness 703 of less than 100 μm and features 701 having a cross-section 762, wherein all corners can be curves.

In an aspect of the present disclosure, reinforced membrane 700, has a silicone membrane 113 having a thickness 703 of less than 100 μm and features 701 having a cross-section 763, with a length 713 of less than 2500 μm, a length 714 of less than 2500 μm, a length 715 of less than 2500 μm, and a radius of length 718 of less than 1250. In certain aspects, features 701 have a cross-section 763, with a length 713 of less than 1000 μm, a length 714 of less than 1000 μm, a length 715 of less than 1000 μm, and a radius of length 718 of less than 500. In certain aspects, features 701 have a cross-section 763, with a length 713 of less than 500 μm, a length 714 of less than 500 μm, a length 715 of less than 500 μm, and a radius of length 718 of less than 250 μm. In other aspects, features 701 have a cross-section 763, with a length 713 of between 100 μm and 2500 μm, a length 714 of between 100 μm and 2500 μm, a length 715 of between 100 μm and 2500 μm, and a radius of length 718 of less than length 714. In yet other aspects, features 701 have a cross-section 763, with a length 713 of between 100 μm and 2500 μm, a length 714 of between 100 μm and 2500 μm, a length 715 of between 100 μm and 2500 μm, and a radius of length 718 equal to length 714. In other aspects, features 701 have a cross-section 763, with a length 713 of between 250 μm and 1000 μm, a length 714 of between 250 μm and 1000 μm, a length 715 of between 250 μm and 1000 μm, and a radius of length 718 of between 5 and 1000 μm. In yet other aspects, features 701 have a cross-section 763, with a length 713 of between 250 μm and 1000 μm, a length 714 of between 250 μm and 1000 μm, a length 715 equal to length 713 and a radius of length 718 of between 5 and 1000 μm. In a further aspect, features 701 have a cross-section 763, with a length 713 of between 250 μm and 1000 μm, a length 714 of between 250 μm and 1000 μm, a length 715 less than length 713, and a radius of length 718 of between 5 and 1000 μm.

In an aspect of the present disclosure, reinforced membrane 700, has a silicone membrane 113 having a thickness 703 of less than 100 μm and features 701 having a cross-section 764, with a length 713 of less than 2500 μm, a length 714 of less than 2500 μm, a length 715 of less than 2500 μm, and an angle 719 of less than 60°. In certain aspects, features 701 have a cross-section 764, with a length 713 of less than 1000, a length 714 of less than 1000 μm, a length 715 of less than 1000 μm, and an angle 719 of less than 60°. In certain aspects, features 701 have a cross-section 764, with a length 713 of less than 500 μm, a length 714 of less than 500 μm, a length 715 of less than 500 μm, and angle 719 of less than 60°. In other aspects, features 701 have a cross-section 764, with a length 713 of between 100 μm and 2500 μm, a length 714 of between 100 μm and 2500 μm, a length 715 of between 100 μm and 2500 μm, and an angle 719 of between 20° and 60°. In other aspects, features 701 have a cross-section 764, with a length 713 of between 250 μm and 1000 μm, a length 714 of between 250 μm and 1000 μm, a length 715 of between 250 μm and 1000 μm, and an angle 719 of between 20° and 60°.

In an aspect of the present disclosure, reinforced membrane 700, has a silicone membrane 113 having a thickness 703 of less than 100 μm and features 701 having a cross-section 765, with a length 713 of less than 2500 μm, a length 714 of less than 2500 μm, a length 715 of less than 2500 μm, and a radius of length 718 of less than 1250 μm. In certain aspects, features 701 have a cross-section 765, with a length 713 of less than 1000 μm, a length 714 of less than 1000 μm, a length 715 of less than 1000 μm, and a radius of length 718 of less than 500. In certain aspects, features 701 have a cross-section 765, with a length 713 of less than 500, a length 714 of less than 500 μm, a length 715 of less than 500 μm, and a radius of length 718 of less than 250 μm. In other aspects, features 701 have a cross-section 765, with a length 713 of between 100 μm and 2500 μm, a length 714 of between 100 μm and 2500 μm, a length 715 of between 100 μm and 2500 μm, and a radius of length 718 of less than length 714. In yet other aspects, features 701 have a cross-section 765, with a length 713 of between 100 μm and 2500 μm, a length 714 of between 100 μm and 2500 μm, a length 715 of between 100 μm and 2500 μm, and a radius of length 718 equal to length 714. In other aspects, features 701 have a cross-section 765, with a length 713 of between 250 μm and 1000 μm, a length 714 of between 250 μm and 1000 μm, a length 715 of between 250 μm and 1000 μm, and a radius of length 718 of between 5 and 500 μm. In yet other aspects, features 701 have a cross-section 765, with a length 713 of between 250 μm and 1000 μm, a length 714 of between 250 μm and 1000 μm, a length 715 equal to length 713 and a radius of length 718 of between 5 and 500 μm. In a further aspect, features 701 have a cross-section 765, with a length 713 of between 250 μm and 1000 μm, a length 714 of between 250 μm and 1000 μm, a length 715 less than length 713, and a radius of length 718 of between 5 and 500 μm.

In an aspect of the present disclosure, reinforced membrane 700, has a silicone membrane 113 having a thickness 703 of less than 100 μm and features 701 having a cross-section 766, with a length 713 of less than 2500 μm, a length 714 of less than 2500 μm, and an angle 719 of less than 60°. In certain aspects, features 701 have a cross-section 766, with a length 713 of less than 1000 μm, a length 714 of less than 1000 μm, and an angle 719 of less than 60°. In certain aspects, features 701 have a cross-section 766, with a length 713 of less than 500, a length 714 of less than 500 μm, and angle 719 of less than 60°. In other aspects, features 701 have a cross-section 766, with a length 713 of between 100 μm and 2500 μm, a length 714 of between 100 μm and 2500 μm, and an angle 719 of between 20° and 60°. In other aspects, features 701 have a cross-section 766, with a length 713 of between 250 μm and 1000 μm, a length 714 of between 250 μm and 1000 μm, and an angle 719 of between 20° and 60°. In yet other aspects, features 701 have a cross-section 766, with a length 713 of between 250 μm and 1000 μm, a length 714 of between 250 μm and 1000 μm, an angle 719 of between 20° and 60°, and a length 715 of less than the length 713.

In an aspect of the present disclosure, reinforced membrane 700, has a silicone membrane 113 having a thickness 703 of less than 100 μm and features 701 having a cross-section 767, with a length 713 of less than 2500 μm, a length 714 of less than 2500 μm, a length 715 less than length 713, and two radii of length 718 of less than 1250 μm each. In certain aspects, features 701 have a cross-section 767, with a length 713 of less than 1000, a length 714 of less than 1000 μm, a length 715 less than length 713, and two radii of length 718 of less than 500 μm each. In certain aspects, features 701 have a cross-section 767, with a length 713 of less than 500 μm, a length 714 of less than 500 μm, a length 715 less than length 713, and two radii of length 718 of less than 250 μm each. In other aspects, features 701 have a cross-section 767, with a length 713 of between 100 μm and 2500 μm, a length 714 of between 100 μm and 2500 μm, a length 715 less than length 713, and two radii of length 718 of less than length 714.

The present disclosure provides for and includes, a reinforced membrane 700, having a feature 701 with length 713 of less than 10000 μm, perpendicular to length 714 of less than 5000 μm. In certain aspects, feature 701 has a length 713 of less than 2500 μm, perpendicular to length 714 of less than 2500 μm. In other aspects, feature 701 has a length 713 of less than 1000 μm, perpendicular to length 714 of less than 1000 μm. In other aspects, feature 701 has a length 713 of less than 500 μm, perpendicular to length 714 of less than 500 μm. In another aspect, feature 701 has a length 713 of between 200 and 5000 μm, perpendicular to length 714 of between 20 and 5000 μm. In a further aspect, feature 701 has a length 713 of between 300 and 800 μm, perpendicular to length 714 of between 20 and 5000 μm. In another aspect, feature 701 has a length 713 of between 1000 and 5000 μm, perpendicular to length 714 of between 20 and 5000 μm. In certain aspects, feature 701 has a length 713 of between 5000 and 8000 μm, perpendicular to length 714 of between 20 and 5000 μm. In other aspects, feature 701 has a length 713 of between 500 and 2500 μm, perpendicular to length 714 of between 20 and 5000 μm. In yet another aspect, feature 701 has a length 713 selected from the group consisting of between 200 and 5000 μm, between 300 and 800 μm, between 1000 and 5000 μm, and between 5000 and 8000 μm, perpendicular to length 714 selected from the group consisting of between 20 and 100 μm, between 20 and 5000 μm, between 100 and 2000 μm, between 100 and 500 μm, between 500 and 1000 μm, 1500 and 2000 μm, between 2000 and 3500 μm, and between 3500 and 5000 μm. In yet other aspects, feature 701 has a length 713, perpendicular to length 714, wherein length 713 is equal to length 714. In certain aspects, feature 701 has a length 713, perpendicular to length 714, wherein length 713 is greater than length 714. In certain aspects, feature 701 has a length 713, perpendicular to length 714, wherein length 713 is less than length 714.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 720. Non-limiting examples of selected patterns suitable for use in a reinforced membrane 700 are illustrated in FIG. 8. As patterns for tiling a Euclidian plane are known in the art, it is understood that other patterns 720 may be incorporated into a reinforced membrane 700 wherein the area 702 comprises at least 70% of the surface and the features 701 comprise 30% or less of the surface. In aspects according to the present disclosure, pattern 720 is selected from the group consisting of patterns 721 to 732, and 733, as illustrated in FIG. 8. As noted above, features 701 provide for mechanical support and strengthen the membrane to reduce tearing splitting, or rupturing. In some aspects, the pattern 720 is a regular tiling pattern, for example patterns 721, 722, 728, or 730, and as illustrated at FIGS. 8A, 8B, 8H, and 8K. Regular tiling patterns having regular polygons that are all the same (e.g., triangle, square, hexagon) are known in the art. In other aspects, pattern 720 is a uniform tiling (also known as a semi-regular tiling) that comprises a mixture of different regular polygons. Uniform tilings are well known in the art. In yet other aspects, the pattern 720 is a non-regular, non-uniform pattern, for example as illustrated in FIG. 8N, pattern 733. As will be appreciated by a person of skill in the art, an very large number of possible patterns 720 are available for use in a reinforced membrane 700, limited by the requirements of area 702 and providing suitable oxygen permeability.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 721 wherein length 711 is between 2 millimeters (mm) and 72 mm and length 712 is between 2 mm and 72 mm. In an aspect, pattern 721 comprises a length 711 between 4 millimeters (mm) and 72 mm and length 712 is between 4 mm and 72 mm. In an aspect, pattern 721 comprises a length 711 between 9 millimeters (mm) and 72 mm and length 712 is between 9 mm and 72 mm. In an aspect, pattern 721 comprises a length 711 between 18 mm and 72 mm and length 712 is between 18 mm and 72 mm. In an aspect, pattern 721 comprises a length 711 between 27 millimeters (mm) and 72 mm and length 712 is between 27 mm and 72 mm. In an aspect, pattern 721 comprises a length 711 between 36 mm and 72 mm and length 712 is between 36 mm and 72 mm. In an aspect, pattern 721 comprises a length 711 between 63 mm and 72 mm and length 712 is between 63 mm and 72 mm. In other aspects, pattern 721 comprises a length 711 and a length 712 of 9 mm. In other aspects, pattern 721 comprises a length 711 and a length 712 of 18 mm. In other aspects, pattern 721 comprises a length 711 and a length 712 of 18 mm. In other aspects, pattern 721 comprises a length 711 and a length 712 of 27 mm. In other aspects, pattern 721 comprises a length 711 and a length 712 of 36 mm. In other aspects, pattern 721 comprises a length 711 and a length 712 of 63 mm. As used herein, pattern 721 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 722 wherein length 711 is between 2 millimeters (mm) and 72 mm. In an aspect, pattern 722 comprises a length 711 between 4 millimeters (mm) and 72 mm. In an aspect, pattern 722 comprises a length 711 between 9 millimeters (mm) and 72 mm. In an aspect, pattern 722 comprises a length 711 between 18 mm and 72 mm. In an aspect, pattern 722 comprises a length 711 between 27 millimeters (mm) and72 mm. In an aspect, pattern 722 comprises a length 711 between 36 mm and 72 mm. In an aspect, pattern 722 comprises a length 711 between 63 mm and 72 mm. In other aspects, pattern 722 comprises a length 711 of 9 mm. In other aspects, pattern 722 comprises a length 711 of 18 mm. In other aspects, pattern 722 comprises a length 711 of 27 mm. In other aspects, pattern 722 comprises a length 711 of 36 mm. In other aspects, pattern 722 comprises a length 711 of 63 mm. As used herein, pattern 722 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 723 wherein the radius of length 718 is between 2 millimeters (mm) and 72 mm. In an aspect, pattern 723 comprises a radius of length 718 between 4 millimeters (mm) and 72 mm. In an aspect, pattern 723 comprises a radius of length 718 between 9 millimeters (mm) and 72 mm. In an aspect, pattern 723 comprises a radius of length 718 between 18 mm and 72 mm. In an aspect, pattern 723 comprises a radius of length 718 between 27 millimeters (mm) and 72 mm. In an aspect, pattern 723 comprises a radius of length 718 between 36 mm and 72 mm. In an aspect, pattern 723 comprises a radius of length 718 between 63 mm and 72 mm.

In other aspects, pattern 723 comprises a radius of length 718 of 9 mm. In other aspects, pattern 723 comprises a radius of length 718 of 18 mm. In other aspects, pattern 723 comprises a radius of length 718 of 27 mm. In other aspects, pattern 723 comprises a radius of length 718 of 36 mm. In other aspects, pattern 723 comprises a radius of length 718 of 63 mm. As used herein, pattern 723 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 724 wherein length 711, the distance between features 701, is between 2 millimeters (mm) and 72 mm. In an aspect, pattern 724 comprises a length 711 between 4 millimeters (mm) and 72 mm. In an aspect, pattern 724 comprises a length 711 between 9 millimeters (mm) and 72 mm. In an aspect, pattern 724 comprises a length 711 between 18 mm and 72 mm. In an aspect, pattern 724 comprises a length 711 between 27 millimeters (mm) and 72 mm. In an aspect, pattern 724 comprises a length 711 between 36 mm and 72 mm. In an aspect, pattern 724 comprises a length 711 between 63 mm and 72 mm. In other aspects, pattern 724 comprises a length 711 of 9 mm. In other aspects, pattern 724 comprises a length 711 of 18 mm. In other aspects, pattern 724 comprises a length 711 of 27 mm. In other aspects, pattern 724 comprises a length 711 of 36 mm. In other aspects, pattern 722 comprises a length 711 of 63 mm. As used herein, pattern 724 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 725, wherein length 711 is between 2 millimeters (mm) and 72 mm, wavelength 716 is between 5 mm and 200 mm, and amplitude 717 is between 5 mm and 72 mm. In an aspect, pattern 725 has a length 711 of between 4 millimeters (mm) and 72 mm, wavelength 716 of between 5 and 200 mm, and amplitude 717 of between 5 and 72 mm. In an aspect, pattern 725 has a length 711 of between 9 millimeters (mm) and 72 mm, wavelength 716 of between 5 and 200 mm, and amplitude 717 of between 5 and 72 mm. In an aspect, pattern 725 has a length 711 of between 27 millimeters (mm) and 72 mm, wavelength 716 of between 5 and 200 mm, and amplitude 717 of between 5 and 72 mm. In an aspect, pattern 725 has a length 711 of between 36 millimeters (mm) and 72 mm, wavelength 716 of between 5 and 200 mm, and amplitude 717 of between 5 and 72 mm. The a reinforced membrane 700 of pattern 725, wherein length 711 is selected from the group consisting of 9 millimeters (mm), 18 mm, 27 mm, 36 mm, and 63 mm. As used herein, pattern 725 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 726 comprising a tile 710 wherein length 711 is between 2 millimeters (mm) and 72 mm and length 712 is between 2 mm and 72 mm. In an aspect, pattern 726 comprises a length 711 between 4 millimeters (mm) and 72 mm and length 712 is between 4 mm and 72 mm. In an aspect, pattern 726 comprises a length 711 between 9 millimeters (mm) and 72 mm and length 712 is between 9 mm and 72 mm. In an aspect, pattern 726 comprises a length 711 between 18 mm and 72 mm and length 712 is between 18 mm and 72 mm. In an aspect, pattern 726 comprises a length 711 between 27 millimeters (mm) and 72 mm and length 712 is between 27 mm and 72 mm. In an aspect, pattern 726 comprises a length 711 between 36 mm and 72 mm and length 712 is between 36 mm and 72 mm. In an aspect, pattern 726 comprises a length 711 between 63 mm and 72 mm and length 712 is between 63 mm and 72 mm. In other aspects, pattern 726 comprises a length 711 and a length 712 of 9 mm. In other aspects, pattern 726 comprises a length 711 and a length 712 of 18 mm. In other aspects, pattern 726 comprises a length 711 and a length 712 of 18 mm. In other aspects, pattern 726 comprises a length 711 and a length 712 of 27 mm. In other aspects, pattern 726 comprises a length 711 and a length 712 of 36 mm. In other aspects, pattern 726 comprises a length 711 and a length 712 of 63 mm. As used herein, pattern 726 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 727 wherein angle 719 is less than 60°, length 711 is between 2 millimeters (mm) and 72 mm and length 712 is between 2 mm and 72 mm. In an aspect, pattern 727 comprises a length 711 between 4 millimeters (mm) and 72 mm and length 712 is between 4 mm and 72 mm. In an aspect, pattern 727 comprises a length 711 between 9 millimeters (mm) and 72 mm and length 712 is between 9 mm and 72 mm. In an aspect, pattern 727 comprises a length 711 between 18 mm and 72 mm and length 712 is between 18 mm and 72 mm. In an aspect, pattern 727 comprises a length 711 between 27 millimeters (mm) and 72 mm and length 712 is between 27 mm and 72 mm. In an aspect, pattern 727 comprises a length 711 between 36 mm and 72 mm and length 712 is between 36 mm and 72 mm. In an aspect, pattern 727 comprises a length 711 between 63 mm and 72 mm and length 712 is between 63 mm and 72 mm. In other aspects, pattern 727 comprises a length 711 and a length 712 of 9 mm. In other aspects, pattern 727 comprises a length 711 and a length 712 of 18 mm. In other aspects, pattern 727 comprises a length 711 and a length 712 of 18 mm. In other aspects, pattern 727 comprises a length 711 and a length 712 of 27 mm. In other aspects, pattern 727 comprises a length 711 and a length 712 of 36 mm. In other aspects, pattern 727 comprises a length 711 and a length 712 of 63 mm. As used herein, pattern 727 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 728 wherein angle 719 is less than 90°, length 711 is between 2 millimeters (mm) and 72 mm and length 712 is between 2 mm and 72 mm. In an aspect, pattern 728 comprises a length 711 between 4 mm and 72 mm and length 712 is between 4 mm and 72 mm. In an aspect, pattern 728 comprises a length 711 between 9 mm and 72 mm and length 712 is between 9 mm and 72 mm. In an aspect, pattern 728 comprises a length 711 between 18 mm and 72 mm and length 712 is between 18 mm and 72 mm. In an aspect, pattern 728 comprises a length 711 between 27 mm and 72 mm and length 712 is between 27 mm and 72 mm. In an aspect, pattern 728 comprises a length 711 between 36 mm and 72 mm and length 712 is between 36 mm and 72 mm. In an aspect, pattern 728 comprises a length 711 between 63 mm and 72 mm and length 712 is between 63 mm and 72 mm. In other aspects, pattern 728 comprises a length 711 and a length 712 of 9 mm. In other aspects, pattern 728 comprises a length 711 and a length 712 of 18 mm. In other aspects, pattern 728 comprises a length 711 and a length 712 of 18 mm. In other aspects, pattern 728 comprises a length 711 and a length 712 of 27 mm. In other aspects, pattern 728 comprises a length 711 and a length 712 of 36 mm. In other aspects, pattern 728 comprises a length 711 and a length 712 of 63 mm. As used herein, pattern 728 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 728 wherein angle 719 is 90°, length 711 is between 2 millimeters (mm) and 72 mm and length 712 is between 2 mm and 72 mm. In an aspect, pattern 728 comprises a length 711 between 4 mm and 72 mm and length 712 is between 4 mm and 72 mm. In an aspect, pattern 728 comprises a length 711 between 9 mm and 72 mm and length 712 is between 9 mm and 72 mm. In an aspect, pattern 728 comprises a length 711 between 18 mm and 72 mm and length 712 is between 18 mm and 72 mm. In an aspect, pattern 728 comprises a length 711 between 27 mm and 72 mm and length 712 is between 27 mm and 72 mm. In an aspect, pattern 728 comprises a length 711 between 36 mm and 72 mm and length 712 is between 36 mm and 72 mm. In an aspect, pattern 728 comprises a length 711 between 63 mm and 72 mm and length 712 is between 63 mm and 72 mm. In other aspects, pattern 728 comprises a length 711 and a length 712 of 9 mm. In other aspects, pattern 728 comprises a length 711 and a length 712 of 18 mm. In other aspects, pattern 728 comprises a length 711 and a length 712 of 18 mm. In other aspects, pattern 728 comprises a length 711 and a length 712 of 27 mm. In other aspects, pattern 728 comprises a length 711 and a length 712 of 36 mm. In other aspects, pattern 728 comprises a length 711 and a length 712 of 63 mm. As used herein, pattern 728 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 729 wherein length 711 is between 2 millimeters (mm) and 72 mm and length 712 is between 2 mm and 72 mm. In an aspect, pattern 729 comprises a length 711 between 4 mm and 72 mm and length 712 is between 4 mm and 72 mm. In an aspect, pattern 729 comprises a length 711 between 9 mm and72 mm and length 712 is between 9 mm and 72 mm. In an aspect, pattern 729 comprises a length 711 between 18 mm and 72 mm and length 712 is between 18 mm and 72 mm. In an aspect, pattern 729 comprises a length 711 between 27 mm and 72 mm and length 712 is between 27 mm and 72 mm. In an aspect, pattern 729 comprises a length 711 between 36 mm and 72 mm and length 712 is between 36 mm and 72 mm. In an aspect, pattern 729 comprises a length 711 between 63 mm and 72 mm and length 712 is between 63 mm and 72 mm. In other aspects, pattern 729 comprises a length 711 and a length 712 of 9 mm. In other aspects, pattern 729 comprises a length 711 and a length 712 of 18 mm. In other aspects, pattern 729 comprises a length 711 and a length 712 of 18 mm. In other aspects, pattern 729 comprises a length 711 and a length 712 of 27 mm. In other aspects, pattern 729 comprises a length 711 and a length 712 of 36 mm. In other aspects, pattern 729 comprises a length 711 and a length 712 of 63 mm. As used herein, pattern 729 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 730 comprising a tile 710 wherein length 711 is between 2 millimeters (mm) and 72 mm and length 712 is between2 mm and 72 mm. In an aspect, pattern 730 comprises a length 711 between 4 millimeters (mm) and 72 mm and length 712 is between4 mm and 72 mm. In an aspect, pattern 730 comprises a length 711 between 9 millimeters (mm) and 72 mm and length 712 is between 9 mm and 72 mm. In an aspect, pattern 730 comprises a length 711 between 18 mm and 72 mm and length 712 is between 18 mm and 72 mm. In an aspect, pattern 730 comprises a length 711 between 27 millimeters (mm) and 72 mm and length 712 is between 27 mm and 72 mm. In an aspect, pattern 730 comprises a length 711 between 36 mm and 72 mm and length 712 is between 36 mm and 72 mm. In an aspect, pattern 730 comprises a length 711 between 63 mm and 72 mm and length 712 is between 63 mm and 72 mm. In other aspects, pattern 730 comprises a length 711 and a length 712 of9 mm. In other aspects, pattern 730 comprises a length 711 and a length 712 of 18 mm. In other aspects, pattern 730 comprises a length 711 and a length 712 of 18 mm. In other aspects, pattern 730 comprises a length 711 and a length 712 of 27 mm. In other aspects, pattern 730 comprises a length 711 and a length 712 of 36 mm. In other aspects, pattern 730 comprises a length 711 and a length 712 of 63 mm. As used herein, pattern 730 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 731 comprising a tile 710 wherein length 711 is between 2 millimeters (mm) and 72 mm and length 712 is between 2 mm and 72 mm. In an aspect, pattern 731 comprises a length 711 between 4 millimeters (mm) and 72 mm and length 712 is between 4 mm and 72 mm. In an aspect, pattern 731 comprises a length 711 between 9 millimeters (mm) and 72 mm and length 712 is between9 mm and 72 mm. In an aspect, pattern 731 comprises a length 711 between 18 mm and 72 mm and length 712 is between 18 mm and 72 mm. In an aspect, pattern 731 comprises a length 711 between 27 millimeters (mm) and 72 mm and length 712 is between 27 mm and 72 mm. In an aspect, pattern 731 comprises a length 711 between 36 mm and 72 mm and length 712 is between 36 mm and 72 mm. In an aspect, pattern 731 comprises a length 711 between 63 mm and 72 mm and length 712 is between 63 mm and 72 mm. In other aspects, pattern 731 comprises a length 711 and a length 712 of 9 mm. In other aspects, pattern 731 comprises a length 711 and a length 712 of 18 mm. In other aspects, pattern 731 comprises a length 711 and a length 712 of 18 mm. In other aspects, pattern 731 comprises a length 711 and a length 712 of 27 mm. In other aspects, pattern 731 comprises a length 711 and a length 712 of 36 mm. In other aspects, pattern 731 comprises a length 711 and a length 712 of 63 mm. As used herein, pattern 731 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 732 comprising a tile 710 wherein length 711 is between 2 millimeters (mm) and 72 mm and length 712 is between 2 mm and 72 mm. In an aspect, pattern 732 comprises a length 711 between 4 millimeters (mm) and 72 mm and length 712 is between 4 mm and 72 mm. In an aspect, pattern 732 comprises a length 711 between 9 millimeters (mm) and 72 mm and length 712 is between 9 mm and 72 mm. In an aspect, pattern 732 comprises a length 711 between 18 mm and 72 mm and length 712 is between 18 mm and 72 mm. In an aspect, pattern 732 comprises a length 711 between 27 millimeters (mm) and 72 mm and length 712 is between 27 mm and 72 mm. In an aspect, pattern 732 comprises a length 711 between 36 mm and 72 mm and length 712 is between 36 mm and 72 mm. In an aspect, pattern 732 comprises a length 711 between 63 mm and 72 mm and length 712 is between 63 mm and 72 mm. In other aspects, pattern 732 comprises a length 711 and a length 712 of 9 mm. In other aspects, pattern 732 comprises a length 711 and a length 712 of 18 mm. In other aspects, pattern 732 comprises a length 711 and a length 712 of 18 mm. In other aspects, pattern 732 comprises a length 711 and a length 712 of 27 mm. In other aspects, pattern 732 comprises a length 711 and a length 712 of 36 mm. In other aspects, pattern 732 comprises a length 711 and a length 712 of 63 mm. As used herein, pattern 732 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 with a cross-section 760, having a pattern 733. Referring to FIG. 8N, pattern 733 comprises a random arrangement of features 701 having a length 711 that is the average of the lengths of random features 701. Accordingly, area 702 comprises the area of silicone membrane 113, minus the product of the length 711, the width of length 713, and the number of features 701 (product 770). As provided herein, area 702 is the difference between the total area of silicone membrane 113 and product 770. In an aspect, length 711 of pattern 733 is between 2 millimeters (mm) and 72 mm. In an aspect, pattern 733 comprises a length 711 between 4 millimeters (mm) and 72 mm. In an aspect, pattern 733 comprises a length 711 between 9 millimeters (mm) and 72 mm. In an aspect, pattern 733 comprises a length 711 between 18 mm and 72 mm. In an aspect, pattern 733 comprises a length 711 between 27 millimeters (mm) and 72 mm. In an aspect, pattern 733 comprises a length 711 between 36 mm and 72 mm. In an aspect, pattern 733 comprises a length 711 between 63 mm and 72 mm. In other aspects, pattern 733 comprises a length 711 of 9 mm. In other aspects, pattern 733 comprises a length 711 of 18 mm. In other aspects, pattern 733 comprises a length 711 of 18 mm. In other aspects, pattern 733 comprises a length 711 of 27 mm. In other aspects, pattern 733 comprises a length 711 of 36 mm. In other aspects, pattern 733 comprises a length 711 of 63 mm. As used herein, pattern 733 can comprise a feature 701 with a cross-section 760 selected from the group consisting of 761 to 767.

The present disclosure provides for, and includes, a reinforced membrane 700, having silicone membrane 113 with an average thickness 703 of less than 100×10⁻⁶ M (μm) in area 702, reinforced with features 701 having an average peak load of between 0.14 newton (N) and 1.9 N at a deflection of 12.5 mm. In an aspect, the average peak load is 0.4 N±0.05 N at a deflection of 12.5 mm.

The present disclosure provides for, and includes, a reinforced membrane 700, having silicone membrane 113 with an average thickness 703 of less than 100×10⁻⁶ M (μm) in area 702, reinforced with features 701 having an average peak load of between 0.14 newton (N) and 1.9 N at a deflection of 12.5 mm wherein the peak load is maintained for a period of 90 minutes when the load is applied at 1.2 hertz (Hz).

The present disclosure provides for, and includes, a reinforced membrane 700, having silicone membrane 113 with an average thickness 703 of less than 100×10⁻⁶ M (μm) in area 702, reinforced with features 701 wherein the average peak load is decreased between 150 and 350% compared to said silicone membrane 113 lacking said features 701.

The present disclosure provides for, and includes, a reinforced membrane 700, having silicone membrane 113 with an average thickness 703 of less than 100×10⁻⁶ M (μm) in area 702, reinforced with features 701 wherein the average peak load is decreased between 200 and 350% compared to said silicone membrane 113 lacking said feature 701.

The present disclosure provides for, and includes, methods for manufacturing reinforced membranes 700 having silicone membrane 113 with an average thickness 703 of less than 100×10⁻⁶ M (μm) in area 702, reinforced with features 701 with a cross-section 760, having a pattern 720. In certain aspects, the methods of manufacture provide for the preparation of inner collapsible containers 102 comprising a reinforced membrane 700 in a single manufacturing step. The present disclosure provides for, and includes, for methods of manufacturing a reinforced silicone membrane 700 include knife coating, calendaring, compression molding, and injection molding. In an aspect, the method of preparing a silicone membrane 113 having a thickness of less than 100×10⁻⁶ M (μm) comprises knife coating.

The present disclosure provides for, and includes, an injection molded collapsible blood container 102 comprising a silicone membrane 700 comprising a silicone membrane 113 having an area 702 and an average thickness 703 of less than 100×10⁻⁶ M (μm) and a feature 701 on at least one side of said silicone membrane 113 having an average length 713 of between 100 μm and 10000 μm perpendicular to an average length 714 of between 20 μm and 5000 μm. In an aspect, the average thickness 703 of said area 702 is less than 50 μm thick. In another aspect, the average thickness 703 of said area 702 is less than 40 μm thick. In another aspect, the average thickness 703 of said area 702 is less than 30 μm thick. In another aspect, the average thickness 703 of said area 702 is less than 20 μm thick.

In another aspect, an injection molded collapsible blood container 102 comprising a silicone membrane 700 comprising a silicone membrane 113 having an area 702 and an average thickness 703 of between 5 and 100 μm thick. In some aspects, the area 702 has an average thickness 703 of between 5 and 75 μm thick. In additional aspects, the area 702 has an average thickness 703 of between 5 and 50 μm thick. In an aspect, the area 702 has an average thickness 703 of between 5 and 40 μm thick. In an aspect, the area 702 has an average thickness 703 of between 5 and 30 μm thick. In yet another aspect, the area 702 has an average thickness 703 of between 5 and 20 μm thick.

As provided for, and included in the present disclosure, an injection molded collapsible blood container 102 is sealed at the open end with Liquid Silicone Rubber (LSR), high consistency rubber (HCR), or a thermoplastic clip. In an aspect, the injection molded collapsible blood container 102 further includes at least one inlet/outlet 130. In certain aspects, at least two inlet/outlets 130 are included in the collapsible blood container 102. In certain aspects, the one or more inlet/outlets 130 can be incorporated into the blow mold. In other aspects, the one or more inlet/outlets 130 can be incorporated into blow molded collapsible blood container 102 when the open end of the container is sealed.

The present disclosure provides for, and includes, an injection molded collapsible blood container 102 that empties without leakage within 2 minutes under an internal pressure of 50 kilopascals (kPa) above standard atmospheric pressure between two plates.

The present disclosure provides for, and includes, methods for manufacturing reinforced membranes 700 comprising preparing a silicone membrane 113 having a thickness of less than 100×10⁻⁶ M (μm), applying a raised feature 701 having an average length 713 of between 100 μm and 10000 μm perpendicular to an average length 714 of between 20 μm and 5000 μm to the surface of said silicone membrane 113, and curing the resulting silicone membrane 700. In an aspect, the method is a continuous process wherein the preparing a silicone membrane 113 having a thickness of less than 100×10⁻⁶ M (μm) and applying a raised feature 701 occurs in a single process step.

In an aspect, the manufacturing method comprises calendaring silicone between a first and second surface having at least one embossing feature 701. In an aspect, the first surface comprises a roller having a recessed feature 701 arranged in a pattern 720 and the second surface is a conveyor. In an aspect, the method of calendaring comprises a first surface comprising a roller having a first recessed feature 701 arranged in a pattern 720 and a second surface comprising a roller having a second recessed feature 701 arranged in a pattern 720.

The following are a list of exemplary embodiments.

Embodiment 1. An oxygen depletion device 10 for depleting oxygen from blood prior to anaerobic storage comprising:
an outer receptacle 101 substantially impermeable to oxygen;
an inner collapsible blood container 102 comprising one or more chambers that are permeable to oxygen; and
an oxygen sorbent 103 situated within said outer receptacle 101.

Embodiment 2. The oxygen depletion device of embodiment 1, wherein said an inner collapsible blood container 102 comprises a reinforced silicone membrane 600.

Embodiment 3. The oxygen depletion device of embodiment 2, wherein said reinforced silicone membrane 600 is reinforced with a fabric selected from the group consisting of polyester, nylon and polyethylene.

Embodiment 4. The oxygen depletion device of embodiments 2 or 3, wherein said reinforced silicone membrane 600 is reinforced with said fabric by surface bonding.

Embodiment 5. The oxygen depletion device of embodiment 4, wherein said surface bonding is to a partially cured silicone sheet.

Embodiment 6. The oxygen depletion device of any one of embodiments 2 to 4, wherein said reinforced silicone membrane 600 is reinforced by applying an uncured silicone mixture to a fabric, applying to a silicone sheet, and curing said applied silicone.

Embodiment 7. The oxygen depletion device of any one of embodiments 2 to 6, wherein said inner collapsible blood container 102 comprises reinforced silicone membrane 600 having a thickness ranging from about 15 μm to about 200 μm.

Embodiment 8. The oxygen depletion device of any one of embodiments 2 to 7, wherein said thickness is selected from the group consisting of 20 μm, 30 μm, 50 μm, 76 μm, and 120 μm.

Embodiment 9. The oxygen depletion device 10 of any one of embodiments 2 to 8, wherein said inner collapsible blood container 102 has a surface area to volume ratio of at least 0.4 centimeters/milliliter (cm²/ml) when filled with blood for depletion and enclosed within said outer receptacle 101.

Embodiment 10. The oxygen depletion device 10 of any one of embodiments 2 to 9, wherein said blood compatible container 102 comprises a material having a permeability to oxygen of at least about 25 Barrer.

Embodiment 11. The oxygen depletion device of any one of embodiments 1 to 10, wherein said oxygen depletion device 10 further comprises a headspace defined by said collapsible blood container 102 and said an outer receptacle 101 substantially impermeable to oxygen, wherein said oxygen sorbent 103 is disposed.

Embodiment 12. The oxygen depletion device of any one of embodiments 2 to 11, further comprising a head space having a volume of between 10 and 1000 ml.

Embodiment 13. The oxygen depletion device of any one of embodiments 1 to 12, wherein said collapsible blood container 102 is manufactured by blow molding or insert molding.

Embodiment 14. The oxygen depletion device of any one of embodiments 2 to 13, wherein said surface area to volume ratio is at least 0.4 cm²/ml, 0.9 cm²/ml or at least 1.5 cm²/ml.

Embodiment 15. The oxygen depletion device of any one of embodiments 2 to 14, wherein said inner collapsible blood container 102 comprises one or more baffles.

Embodiment 16. The oxygen depletion device of any one of embodiments 1 to 15, wherein said inner collapsible blood container 102 comprises at least two chambers that are permeable to oxygen and said chambers are in fluid communication.

Embodiment 17. The oxygen depletion device of any one of embodiments 1 to 16, wherein said at least two chambers that are permeable to oxygen are stacked within said outer receptacle 101.

Embodiment 18. The oxygen depletion device of any one of embodiments 1 to 17, wherein said at least two chambers that are permeable to oxygen are arranged side by side within said outer receptacle 101.

Embodiment 19. The oxygen depletion device of one of embodiments 1 to 18, wherein said inner collapsible blood container 102 comprises two to eight chambers that are permeable to oxygen and said chamber are in fluid communication.

Embodiment 20. The oxygen depletion device of any one of embodiments 1 to 19, wherein said two to eight chambers are arranged side by side within said outer receptacle.

Embodiment 21. The oxygen depletion device of any one of embodiments 1 to 17, wherein said at least one sorbent 103 is positioned between said stacked chambers of said inner collapsible blood container 102.

Embodiment 22. A reinforced silicone membrane comprising a silicone membrane layer of between 5 μm and 100 μm and a fabric layer of between 50 μm to 1.5 mm thick bonded to one side of said silicone membrane layer.

Embodiment 23. The reinforced silicone membrane of embodiment 22, wherein said silicone membrane layer is 14 μm, 25 μm, or 50 μm thick.

Embodiment 24. The reinforced silicone membrane of embodiment 22 or 23, wherein said fabric is selected from the group consisting of polyester, nylon and polyethylene.

Embodiment 25. The reinforced silicone membrane of any one of embodiments 22 to 25, wherein said fabric is coated with a 2 to 10 μm silicone layer.

Embodiment 26. A silicone membrane 700 comprising:

a silicone membrane 113 having an area 702 and an average thickness 703 of less than 100×10⁻⁶ M (μm), and a feature 701 on at least one side of said silicone membrane 113 comprising silicone having an average length 713 of between 100 μm and 10000 μm perpendicular to an average length 714 of between 20 μm and 5000 μm.

Embodiment 27. The silicone membrane 700 of embodiment 26, wherein said raised feature 701 covers a percentage of less than 50% of the area of said at least one side of said silicone membrane 700.

Embodiment 28. The silicone membrane 700 of embodiment 26 or 27, wherein said percentage is between 10 and 50% of the area of said at least one side of said silicone membrane 113.

Embodiment 29. The silicone membrane 700 of any one of embodiments 26 to 28, wherein said average thickness 703 of said area 702 is selected from the group consisting of less than 50 μm thick, less than 40 μm thick, less than 30 μm thick, less than 20 μm thick, and between 5 and 50 μm thick.

Embodiment 30. The silicone membrane 700 of any one of embodiments 26 to 29, wherein said average length 713 is between 200 and 5000 μm or between 500 and 2500 μm.

Embodiment 31. The silicone membrane 700 of any one of embodiments 26 to 30, wherein said average length 714 is between 100 μm and 2000 μm, between 500 μm and 1000 μm, or between 100 μm to 500 μm.

Embodiment 32. The silicone membrane 700 of any one of embodiments 26 to 31, wherein said silicone membrane 700 has an average peak load of between 0.14 newton (N) and 1.9 N at a deflection of 12.5 mm.

Embodiment 33. The silicone membrane 700 of any one of embodiments 26 to 32, wherein said peak load that is between 0.14 newton (N) and 1.9 N at a deflection of 12.5 millimeter (mm) is maintained for a period of 90 minutes when the load is applied at 1.2 hertz (Hz).

Embodiment 34. The silicone membrane 700 of embodiment 26, wherein said raised feature 701 increases the average peak load at 12.5 millimeter (mm) deflection of said silicone membrane 113 by between 150 and 350% compared to said silicone membrane 113 lacking said raised feature.

Embodiment 35. The silicone membrane 700 of any one of embodiments 26 to 34, wherein said raised feature 701 increases said average peak load at 12.5 millimeter (mm) deflection of said silicone membrane 113 by between 200 and 350% compared to said silicone membrane 113 lacking said raised feature.

Embodiment 36. The silicone membrane 700 of any one of embodiments 26 to 35, wherein said silicone membrane 700 has an average peak load of 0.4 N±0.05 N at a deflection of 12.5 mm.

Embodiment 37. The silicone membrane 700 of any one of embodiments 26 to 36, wherein said raised feature 701 comprises cross section 761, 762, 763, 764, 765, 766, 767, or a combination thereof.

Embodiment 38. The silicone membrane 700 of any one of embodiments 26 to 37, wherein said raised feature 701 comprises a cross-section 760 selected from the group consisting of:
a. cross-section 761 comprising a length 714 of between 100 μm and 2500 μm and a radius of length 718 of between 100 μm and 500 μm; ;
b. cross-section 762 comprising a length 713 of between 100 μm and 2500 μm, a length 714 of between 100 μm and 2500 μm, and a length 715 of zero;
c. cross-section 762 comprising a length 713 of between 100 μm and 2500 μm, a length 714 of between 100 μm and 2500 μm, wherein length 715 equals length 713;
d. cross-section 762 comprising a length 713 of between 100 μm and 2500 μm, a length 714 of between 100 μm and 2500 μm, wherein length 715 is greater than length 713;
e. cross-section 763 comprising a length 713 of between 100 μm and 2500 μm, a length 714 of between 100 μm and 2500 μm, and a radius of length 718 between 5 μm and 100 μm wherein length 715 is equal to length 713;
f. cross-section 764 comprising a length 713 of between 100 μm and 2500 μm, a length 714 of between 100 μm and 2500 μm, and an angle 719 between 20° and 60°, wherein length 715 is equal to length 713;
g. cross-section 765 comprising a length 713 of between 100 μm and 2500 μm, a length 714 of between 100 μm and 2500 μm, and a radius of length 718 between 5 μm and 100 μm, wherein length 715 is equal to length 713;
h. cross-section 766 comprising a length 713 of between 100 μm and 2500 μm, a length 714 of between 100 μm and 2500 μm, and an angle 719 of between 20° and 60°; and
i. cross-section 767 comprising a length 713 of between 100 μm and 2500 μm, a length 714 of between 100 μm and 2500 μm, and a radius of length 718 of between 5 μm and 100 μm or between 15 μm and 30 μm and a length 715 of 50 to 0 μm.

Embodiment 39. The silicone membrane 700 of any one of embodiments 26 to 38, wherein said cross section 760 is selected from the group consisting of:
a. cross-section 761 comprising a length 714 of between 250 μm and 1000 μm and a radius of length 718 of between 100 μm and 500 μm;
b. cross-section 762 comprising a length 713 of between 250 μm and 1000 μm, a length 714 of between 250 μm and 1000 μm, and a length 715 of zero;
c. cross-section 762 comprising a length 713 of between 250 μm and 1000 μm, a length 714 of between 250 μm and 1000 μm, wherein length 715 equals length 713;
d. cross-section 762 comprising a length 713 of between 250 μm and 1000 μm, a length 714 of between 250 μm and 1000 μm, wherein length 715 is greater than length 713;
e. cross-section 763 comprising a length 713 of between 250 μm and 1000 μm, a length 714 of between 250 μm and 1000 μm, and a radius of length 718 between 5 μm and 100 μm, wherein length 715 is equal to length 713;
f. cross-section 764 comprising a length 713 of between 250 μm and 1000 μm, a length 714 of between 250 μm and 1000 μm, and an angle 719 between 20° and 60 20°, wherein length 715 is equal to length 713;
g. cross-section 765 comprising a length 713 of between 250 μm and 1000 μm, a length 714 of between 250 μm and 1000 μm, and a radius of length 718 between 5 μm and 100 μm, wherein length 715 is equal to length 713;
h. cross-section 766 comprising a length 713 of between 250 μm and 1000 μm, a length 714 of between 250 μm and 1000 μm, and an angle 719 of between 15 μm and 30 μm; and
i. cross-section 767 comprising a length 713 of between 250 μm and 1000 μm, a length 714 of between 250 μm and 1000 μm, and a radius of length 718 of between 5 μm and 100 μm or between 15 μm and 30 μm and a length 715 of 0 to 50 μm.

Embodiment 40. The silicone membrane 700 of any one of embodiments 26 to 39, wherein said raised feature 701 comprises a pattern 720 selected from the group consisting of
(i) pattern 721, wherein length 711 is between 2 mm and 72 mm, and length 714 is between 4.0 mm and 72 mm;
(ii) pattern 722, wherein length 711 is between 2 mm and 72 mm;
(iii) pattern 723 wherein radius 718 is between 2.0 mm and 72 mm;
(iv) pattern 724, wherein length 711 is between 2 mm and 72 mm;
(v) pattern 725, wherein length 711 is between 2 mm and 72 mm, wavelength 716 is between 5 mm and 200 mm, and amplitude 717 is between 5 and 72 mm;
(vi) pattern 726, wherein unit cell 710 has length 711 is between 2 mm and 72 mm
(vii) pattern 727, wherein length 711 is between 2 mm and 72 mm and length 712 is between 2 mm and 72 mm;
(viii) pattern 728, wherein length 711 is between 2 mm and 72 mm and length 712 is between 2 mm and 72 mm, and angle 719 is less than 90°;
(ix) pattern 728, wherein length 711 is between 2 mm and 72 mm and length 712 is between 2 mm and 72 mm, and angle 719 is 90°;
(x) pattern 729, wherein length 711 is between 2 mm and 72 mm and length 712 is between 2 mm and 72 mm;
(xi) pattern 730, wherein unit cell 710 has length 711 is between 2 mm and 72 mm;
(xii) pattern 731, wherein unit cell 710 has length 711 is between 2 mm and 72 mm and length 712 is between 2 mm and 72 mm;
(xiii) pattern 732, wherein unit cell 710 has length 711 is between 2 mm and 72 mm and length 712 is between 2 mm and 72 mm;
(xiv) pattern 733, wherein length 711 is between 2 mm and 72 mm and is the average length of features 701.

Embodiment 41. The silicone membrane 700 of any one of embodiments 26 to 40, wherein said pattern is selected from the group consisting of:
a. pattern 721 comprising a length 711 of 4 mm, 9 mm, 18 mm, 27 mm, 36 mm, or 63 mm;
b. pattern 722 comprising a length 711 of 4 mm, 9 mm, 18 mm, 27 mm, 36 mm, or 63 mm;
c. pattern 723 comprising a length 711 of 4 mm, 9 mm, 18 mm, 27 mm, 36 mm, or 63 mm;
d. pattern 724 comprising a length 711 of 4 mm, 9 mm, 18 mm, 27 mm, 36 mm, or 63 mm;
e. pattern 725 comprising a length 711 of 4 mm, 9 mm, 18 mm, 27 mm, 36 mm, or 63 mm;
f. pattern 726 comprising a length 711 of 4 mm, 9 mm, 18 mm, 27 mm, 36 mm, or 63 mm;
g. pattern 727 comprising a length 711 of 4 mm, 9 mm, 18 mm, 27 mm, 36 mm, or 63 mm;
h. pattern 728 comprising a length 711 of 4 mm, 9 mm, 18 mm, 27 mm, 36 mm, or 63 mm;
i. pattern 729 comprising a length 711 of 4 mm, 9 mm, 18 mm, 27 mm, 36 mm, or 63 mm;
j. pattern 730 comprising a length 711 of 4 mm, 9 mm, 18 mm, 27 mm, 36 mm, or 63 mm;
k. pattern 731 comprising a length 711 of 4 mm, 9 mm, 18 mm, 27 mm, 36 mm, or 63 mm;
l. pattern 732 comprising a length 711 of 4 mm, 9 mm, 18 mm, 27 mm, 36 mm, or 63 mm; and
m. pattern 733 comprising a length 711 of 4 mm, 9 mm, 18 mm, 27 mm, 36 mm, or 63 mm.

Embodiment 42. The silicone membrane 700 of any one of embodiments 26 to 42, wherein said raised feature 701 comprises a regular tiling pattern, a uniform tiling, or an non-regular, non-uniform pattern.

Embodiment 43. The silicone membrane 700 of embodiment 42, wherein said uniform tiling comprises a triangular tiling, a square tiling, a hexagonal tiling, tri-hexagonal, or prismatic pentagonal tiling.

Embodiment 44. The silicone membrane 700 of embodiments 42 or 43, wherein said silicone membrane 700 is injection molded, compression molded, spray molded, calendered, or cast.

Embodiment 45. The silicone membrane 700 of any one of embodiments 42 to 44, wherein said raised feature 701 is present on a single side of said silicone membrane 113.

Embodiment 46. The silicone membrane 700 of any one of embodiments 42 to 45, wherein said silicone membrane 113 said feature 701 comprises a first feature 701 on a first side and a second feature 701 on a second side.

Embodiment 47. The silicone membrane 700 of any one of embodiments 42 to 46, wherein said second feature 701 is different from said first feature 701.

Embodiment 48. The silicone membrane 700 of any one of embodiments 42 to 47, wherein said silicone membrane 700 has a permeability of at least 1.2×10⁻⁶ ml/sec*cm²*mmHg.

Embodiment 49. A collapsible blood container 102 comprising a silicone membrane 700 according any one of embodiments 1 to 48.

Embodiment 50. The collapsible blood container 102 of embodiment 49, wherein said blood container 102 further comprises a frame 121.

Embodiment 51. The collapsible blood container 102 of embodiment 49 or 50, wherein said frame 121 is a silicone frame.

Embodiment 52. The collapsible blood container 102 of any one of embodiments 40 to 51, wherein said frame 121 comprises high consistency rubber (HCR).

Embodiment 53. An injection molded collapsible blood container 102 comprising a silicone membrane 700 comprising a silicone membrane 113 having an area 702 and an average thickness 703 of less than 100×10⁻⁶ M (μm) and a feature 701 on at least one side of said silicone membrane 113 having an average length 713 of between 100 μm and 10000 μm perpendicular to an average length 714 of between 20 μm and 5000 μm.

Embodiment 54. The injection molded collapsible blood container 102 of embodiment 53, wherein said average thickness 703 of said area 702 is selected from the group consisting of less than 50 μm thick, less than 40 μm thick, less than 30 μm thick, less than 20 μm thick, and between 5 and 50 μm thick.

Embodiment 55. The injection molded collapsible blood container 102 of embodiment 53 or 54, wherein said injection molded blood container 102 is sealed at the open end with Liquid Silicone Rubber (LSR), high consistency rubber (HCR), or a thermoplastic clip.

Embodiment 56. The injection molded collapsible blood container 102 of any one of embodiments 53 to 55, wherein said injection molded blood container 102 further comprises at least one inlet/outlet 130.

Embodiment 57. The injection molded collapsible blood container 102 of any one of embodiments 53 to 56, when filled with water at 23±5° C. equal to its nominal capacity empties without leakage within 2 minutes under an internal pressure of 50 kilopascals (kPa) above standard atmospheric pressure between two plates.

Embodiment 58. A blood filled collapsible blood container 102 comprising a silicone membrane 700 according to any one of embodiments 1 to 57.

Embodiment 59. A method of manufacturing a silicone membrane 700 comprising
(i) preparing silicone membrane 113 having a thickness of less than 100×10⁻⁶ M (μm);
(ii) applying a raised feature 701 having an average length 713 of between 100 μm and 10000 μm perpendicular to an average length 714 of between 20 μm and 5000 μm to the surface of said silicone membrane 113; and
(iii) curing said silicone membrane 700.

Embodiment 60. The method of manufacturing a silicone membrane 700 of embodiment 56, wherein said preparing of step (i) and said applying of step (ii) are a continuous process.

Embodiment 61. The method of manufacturing a silicone membrane 700 of embodiment 59 or 60, wherein said preparing of step (i) comprises knife coating.

Embodiment 62. The method of manufacturing a silicone membrane 700 of any one of embodiments 59 to 61, further comprising said knife coating of said raised feature 701 as a continuous feature.

Embodiment 63. The method of manufacturing a silicone membrane 700 of any one of embodiments 59 to 63, wherein said applying comprises embossing feature 701 by calendaring between a first surface and a second surface.

Embodiment 64. The method of manufacturing a silicone membrane 700 of any one of embodiments 59 to 63, wherein said first surface comprises a roller having a recessed feature 701 arranged in a pattern 720 and said second surface is a conveyor.

Embodiment 65. The method of manufacturing a silicone membrane 700 of any one of embodiments 59 to 64, wherein said first surface comprises a roller having a recessed feature 701 arranged in a pattern 720 and said second surface comprises a roller.

Embodiment 66. The method of manufacturing a silicone membrane 700 of any one of embodiments 59 to 66, wherein said first surface comprises a roller having a first recessed feature 701 arranged in a pattern 720 and said second surface comprises a roller having a second recessed feature 701 arranged in a pattern 720.

Embodiment 67. The method for making a silicone membrane 700 of any one of embodiments 59 to 66, wherein said preparing further includes a step of curing said silicone membrane 113 prior to said applying step (ii).

Embodiment 68. A method of manufacturing a silicone membrane 700, comprising injection molding.

Embodiment 69. The method for manufacturing a silicone membrane 700 of embodiment 68, wherein said continuous feature comprises one or more of a straight line, a zig-zag line, a sinusoidal line, or a combination thereof.

Embodiment 70. The method for manufacturing silicone membrane 700 of embodiment 68 or 69, wherein said sinusoidal line a wavelength 716 of 40 mm and an amplitude 717 of 4 mm.

Embodiment 71. The method for manufacturing a silicone membrane 700 of any one of embodiments 68 to 70, wherein said applying said continuous feature comprises applying said raised feature 701 with a nozzle.

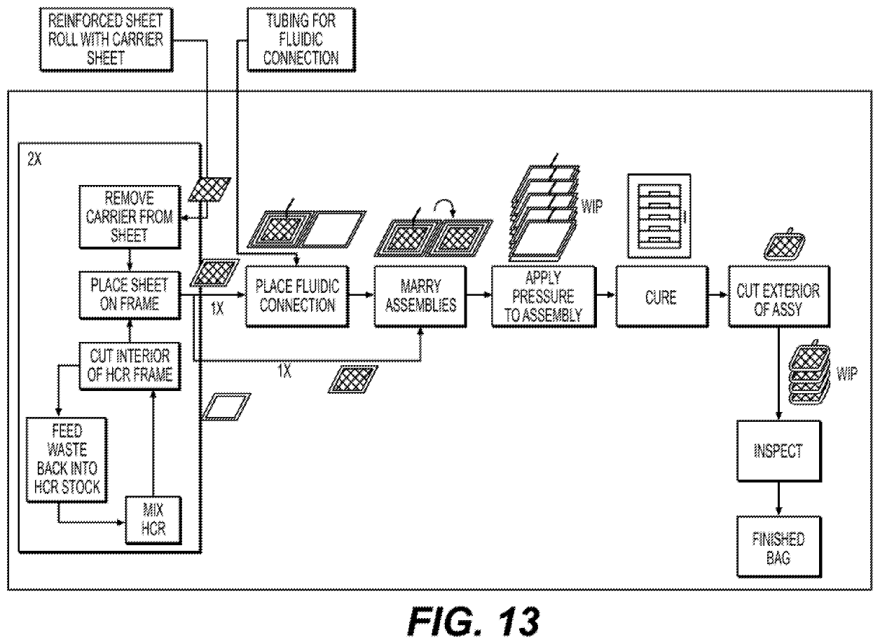

Embodiment 72. The method for manufacturing a silicone membrane 700 of any one of embodiments 58 to 71, comprising the process of FIG. 13 to make a resulting reinforced bag.

While the present disclosure has been described with reference to preferred embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

### EXAMPLES

### Example 1: Fabrication of an Inner Collapsible Blood Container 102, Spot Bonded Reinforced Silicone

A silicone bag is made from a pair of Wacker Silpuran® silicone sheets about 7 inches square and 30 μm thick using Smooth-On Silpoxy® adhesive to seal the perimeter with a piece of ¼″ diameter silicone tubing bonded into the seam for fluid access to the lumen of the bag. The bag is reinforced by bonding two sheets of 4-mm square opening polypropylene mesh (Conwed # R3650) around the periphery on both sides of the bag using Smooth-On Silpoxy® adhesive. The polypropylene mesh on one side of the bag is further bonded with four spots of adhesive about 5×10 mm spaced equally around the center of the bag; the polypropylene mesh on the other side of the bag is further bonded with five spots of adhesive about 5 mm diameter spaced equally around the center of the bag with one spot bond in the center. The bag is filled with water and drop tested from a height of about 3 feet multiple times with no leaks. Drop testing from about 5 feet resulted in bag rupture near the center of the side with 5 spot bonds.

### Example 2: Fabrication of Reinforced Silicone Sheeting Suitable for Use in an Inner Collapsible Blood Container 102, Partially Cured Sheet Method

Silicone sheets having a thickness of about 25 μm are fabricated by mixing one part silicone LSR (liquid silicone rubber) part A with one part silicone LSR part B (NuSil MED 10-6640) and diluted in 10 to 90% w/w xylene. The mixture is spread out and passed under a precision knife edge on a custom built knife coating machine to yield a sheet about 9 inches by 11 inches by about 50 μm thick before solvent evaporation. The sheet is partially cured by heating for about 3-5 minutes at 38-55° C. (estimated) before placing a sheet of polyester mesh fabric (Mohawk Fabrics P/N 400485) onto the partially cured silicone sheet. The polyester mesh fabric is pressed into the sheet and placed back into an oven at 65-121° C. for about 10 minutes to complete the curing of the LSR to yield a fabric reinforced silicone sheet about 9 inches by 11 inches and having an interstitial silicone thickness of about 25μm thick, not inclusive of the polyester mesh fabric reinforcement material. The polyester mesh fabric is adhered to the cured silicone sheet, but is not totally encapsulated by the silicone sheet. The one surface of the reinforced sheets has a matte finish suitable for contact with blood or blood products. Additional reinforced silicone sheets having thicknesses of about 13 μm and about 50 μm are fabricated using the partially cured sheet method.

### Example 3: Fabrication of an Inner Collapsible Blood Container 102, Using Reinforced Silicone Sheeting

To fabricate a blood bag from the fabric reinforced sheets, the fabric reinforced sheets are trimmed to about 9 inches square and a frame made of NuSil MED-4050 silicone HCR (High Consistency Rubber) is placed between the pair of fabric reinforced silicone sheets. Pressure is applied to this assembly while curing in an oven for about 30 minutes at 115-121° C. to yield an oxygen permeable blood bag that had internal dimensions of about 8 inches square. The oxygen permeable blood bag was able to reduce the blood oxygen content of a sample of LRpRBC to below 5% with agitation in the presence of oxygen sorbent within 3 hours.

Reinforced silicone collapsible blood containers 102 according to FIGS. 3 to are prepared having the polyester mesh fabric prepared in Example 3 on the outside surface while the inside surface of the container is a matte finish silicone. The membrane 600 is joined together to form container 102 by a frame 120. Each container includes a tube 121 for introducing and removing blood and other fluids. Containers 102 are tested by inflating with nitrogen to 1 psig and submerged in water for 30 seconds to test for leakage. Only containers that passed the leak test are used in the construction of an ORB.

### Example 4: Fabrication of Reinforced Silicone Sheeting Suitable for Use in an Inner Collapsible Blood Container 102, Fully Cured Sheet Bonding Method

A silicone sheet about 200×250 mm and about 20 μm thick (Wacker Silpuran®) is bonded to a sheet of nylon fabric mesh by mixing one part silicone LSR (liquid silicone rubber) part A with one part silicone LSR part B (Wacker Silpuran® 2030) mixed in about 10 to 90% w/w xylene, applying the silicone LSR mixture to the fabric and pressing the silicone sheet and fabric together with heat until the silicone LSR mixture is fully cured and the solvent is evaporated. A frame made of NuSil SIL2-5070 silicone HCR (High Consistency Rubber) is placed between the two fabric reinforced sheets, placed in a press under pressure and cured in an oven at about 115-121° C. for about 10 minutes to yield a blood bag that is able to reduce the blood oxygen content of a unit of LRpRBC to below 5% with agitation in the presence of oxygen sorbent within 3 hours. Reinforced silicone collapsible blood containers 102 are prepared and tested as described in Example 3.

### Example 5: Drop Test of 8″×8″ Reinforced Inner Collapsible Blood Containers

A reinforced silicone collapsible blood container 102 prepared according to Example 3 is assembled into an Oxygen Reduction Bag having a bed of Dessicare® sorbent (Dessicare Part Number S1200B03, Dessicare, Inc., Reno, Nevada) in a Clearfoil® Z barrier bag as described in the '130 Provisional Application. A total of 4 assemblies are tested after concluding deoxygenation tests. Blood is drained from the ORB assemblies after deoxygenation. The assemblies are then rinsed with saline and filled with 25% glycerol in water to simulate blood density (dyed blue to help visualize potential leaks). The assemblies are then dropped from a height of 6 feet onto a 12″×15″ stainless steel plate in a large biohazard bag within a 16″×28″×13″ high open topped cardboard box. Each ORB assembly is dropped one time only. The assemblies are dropped on the sorbent with the bag above the bed of sorbent, on the silicone bag with the bed of sorbent above the bag, on one corner of the assembly, and on the tube seal. All ORB assemblies pass the drop tests from 6 feet from all four drop configurations.

### Example 6: Oxygen Depletion of Leukoreduced Packed Red Blood Cells Using Oxygen Depletion Devices With Reinforced Silicone Collapsible Blood Containers Having 14 μm, 25 μm, and 50 μm Thick Silicone Membranes

Oxygen depletion devices as described in the '130 Provisional were prepared using an outer receptacle 101 prepared from Clearfoil® Z, a spacer 111 (McMaster Carr #9314T29, NJ McMaster Carr, Inc., Robbinsville, NJ), and a sorbent 103 provided in 15 sachets (Dessicare Part Number S1200B03, Dessicare, Inc., Reno, Nevada) using reinforced membranes 600 having thicknesses of 14 μm, 25 μm, and 50 μm as provided in Table 2.

For each test, four (4) each ORB Test Bags (2×146 mL; 2×110 mL) are prepared having the indicated thickness and pooled type matched leukoreduced packed red blood cells (LR-pRBCs) are generated and oxygenated until the starting SO2 was >90%. A sample bag is sterile docked to the pool bag and a 3.5 mL sample is taken from the pool using a standard 5 ml syringe with a 16 gauge needle and tested for T=0 hemolysis levels using a HemoCue Plasma Analyzer. It is also tested on the ABL-90 for starting SO2 and pCO2 levels. About 146 mL of pRBC product is transferred into two of the ORBs, and 110 mL is transferred into the remaining two bags from the pool after sterile docking them. The source bag is disconnected from each ORB using a tube sealer and a sample bag is sterile docked to each ORB. ORBs are placed horizontally on a Helmer platelet shaker with the port and sampling bag on the left. Each ORB is deoxygenated on the platelet shaker for 3 hours. 1 ml samples are taken at T=0, 30, 60, 120 using a standard 1 ml syringe with a 16 gauge needle and are tested for SO2 and pCO2 levels on an ABL-90. A 3.5 ml sample is taken at T=180 min using a standard 5 ml syringe with a 16 gauge needle and is tested for hemolysis levels on the HemoCue Plasma Analyzer and for SO2 and pCO2 levels on an ABL-90. All samples are taken from the sampling bags sterile docked to each ORB. The results are summarized in Table 2.

TABLE 2
Oxygen depletion using devices having inner blood containers
prepared from reinforced silicone membranes

| | Silicone | | Blood | Initial | SO2 | SO2 | Rate |
|---|---|---|---|---|---|---|---|
| Test bag | Membrane | Dimensions | volume | hematocrit | T = 0 | T = 180 | constant |
| number # | 113 | (inches) | (mL) | (%) | (%) | (%) | (min⁻¹) |
| ORB1 | 13 μm | 7.75 × 7.625 | 149.91 | 50.4 | 98.1 | 6.6 | −1.53E−02 |
| ORB2 | 13 μm | 7.625 × 7.5 | 146.51 | 50.4 | 98.1 | 3.5 | −1.87E−02 |
| ORB3 | 13 μm | 7.375 × 7.75 | 116.04 | 50.3 | 98.1 | 3.8 | −1.82E−02 |
| ORB 4 | 13 μm | 7.5 × 7.25 | 110.47 | 50.4 | 98.2 | 4.3 | −1.74E−02 |
| ORB5 | 25 μm | 6.5 × 7.5 | 149.15 | 49.6 | 98.4 | 5.6 | −1.66E−02 |
| ORB6 | 25 μm | 6.5 × 7.5 | 149.81 | 49.6 | 98.7 | 5.6 | −1.60E−02 |
| ORB7 | 25 μm | 6.5 × 7.5 | 115 | 56.2 | 98.6 | 5.1 | −1.67E−02 |
| ORB8 | 25 μm | 6.5 × 7.5 | 115.66 | 49.3 | 98.8 | 6.7 | −1.55E−02 |
| ORB9 | 25 μm | 10 × 16 | 115.66 | 59.1 | 96.8 | 6.3 | −1.54E−02 |
| ORB10 | 25 μm | 10 × 16 | 115.66 | 59.0 | 97.1 | 8.8 | −1.42E−02 |
| ORB11 | 25 μm | 10 × 16 | 115.66 | 58.5 | 97.5 | 10.3 | −1.40E−02 |
| ORB12 | 50 μm | 7.75 × 7.625 | 149.34 | 56.6 | 98.6 | 9 | −1.48E−02 |
| ORB13 | 50 μm | 7.75 × 7.625 | 149.15 | 56.2 | 100.1 | 10 | −1.29E−02 |
| ORB14 | 50 μm | 7.75 × 7.625 | 111.79 | 56.5 | 100.1 | 4.5 | −1.50E−02 |
| ORB15 | 50 μm | 7.75 × 7.625 | 110.94 | 56.3 | 99 | 7.8 | −1.43E−02 |

### Example 7: Automated Reinforced Silicone Collapsible Blood Container Manufacture From a Roll of Reinforced Silicone Sheet

Referring to FIG. 13, small and large scale reinforced silicone collapsible blood containers may be manufactured. As indicated as item 1, parts A and part B of a High Consistency Rubber (HCR) are mixed together in a two roll mill to create a homogenous high viscosity silicone sheet in an uncured state that is 0.25 mm-0.2.5 mm thick. In item two, an HCR “frame” (frame 120 in FIGS. 3 to 5) is prepared by cutting the forming HCR sheet to size and desired interior shape using a steel rule, rotary style, or equivalent die. Excess material from the interior of the frame is returned to the uncured HCR stock as shown in item 3. Reinforced silicone sheets in roll form with a backing sheet are introduced and the backing removed leaving the silicone exposed (item 4). The reinforced silicone sheet is placed silicone side down, on the HCR frame from item 2 and shown as item 5. One or more subassemblies according to items 1 to 5 can be prepared. As shown in item 6, fluidic connections are introduced on one side of one frame/sheet subassembly from item 5. Suitable fluidic connections can be a silicone tube, a silicone over-molded barb fitting, or a barb fitting made of polycarbonate, nylon, polypropylene or other polymer. Plasma treatment may be optionally included to improve bonding between the fitting and HCR frame. A second frame/sheet subassembly from item 5 is placed on the onto the subassembly with fluidic connection from item 6, with the two HCR frames mated together as shown in item 7. As provided in item 8, pressure is applied to the assembly from step 7, using a press, rollers, or their equivalent at about 20 to 70 newtons (N) pressure, or an appropriate pressure to ensure contact of the assembly. The assembly is placed in an oven, a tunnel kiln, or their equivalent to cure, typically about 10 to 20 minutes at 115-130° C. as shown in item 9. Following curing, the exterior of the assembly is cut to the desired final container shape, using a steel rule, a rotary die or their equivalent as shown in item 10. As shown in item 11, completed containers are inspected for imperfections and for leaks, for example using a pressure decay tool that measures a loss of pressure following filling the container with nitrogen. Multiple cycles of pressurizing, stabilizing, and measuring occur over a cycle time of about of 10-20 seconds are performed. Losses of pressure indicates a leak, thus a failed inspection.

### Example 8: Automated Manufacture of Reinforced Silicone Collapsible Blood Containers

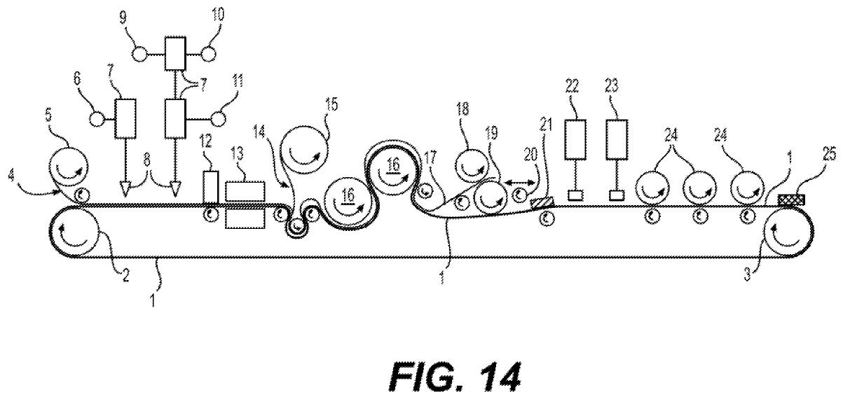

Referring to FIG. 14, continuous process to form a reinforced silicone LSR (Liquid Silicone Rubber) film and further to fabricate a collapsible blood container from the film is provided. In an aspect, the process is driven by a conveyor belt.

The conveyor belt (1) has a drive roller (2) and a take-up roller (3). Optionally, a carrier film (4) may be used on top of the conveyor belt by feeding a polymer film, such as 0.006 inch thick polyester (Mylar®) or PET (Polyethylene terephthalate) from a roll (5) onto the conveyor belt.

At the 1st process station the conveyor belt is treated by spraying a thin layer of silicone release agent using a pump (7) to dispense the material from a reservoir (6) through one or more spray nozzles (8) onto the conveyor belt. Optionally, the conveyor belt (or carrier film) may have a textured surface, wherein such texture is imparted to the silicone LSR film formed thereon and wherein such textured surface prevents the internal surfaces of the bag from sticking to each other and aids in filling the bag with blood.

At the 2nd process station a mixture of silicone LSR is formed by pumping the individual part A and part B of the LSR from their respective reservoirs (9,10), preferably through a mixing chamber (not shown). The uncured LSR mixture is then diluted by the addition of a suitable solvent, such as xylene, by pumping the solvated mixture through a pump (7) from a reservoir (11) and spraying the diluted mixture through one or more spray nozzles (8) onto the conveyor belt (or carrier film) to yield a thin film. The process variables (metering pumps, belt speed, etc.) are adjusted as needed to control the initial film thickness to about 50-75 μm. Optionally, the film thickness can be further controlled by passing under a knife edge (12) to reduce the film thickness to a desired thickness.

The conveyor belt passes through a heat tunnel (13) for a period of about 2-5 minutes at a temperature of about 125-200° F. to partially cure the LSR and partially evaporate the solvent, yielding a LSR/xylene layer having a thickness of about 30-40 μm. Suitable means for heating include infrared lamps, quartz heating rods, and other resistively powered heating elements.

The conveyor belt passes under a roll (15) of polymer mesh (14), such as Mohawk Fabrics polyester P20D, which is spooled from the roll and fed onto the partially cured layer of LSR. Additional rollers may be employed to control the tension as needed to maintain the desired tension on the materials and contact between them.

The conveyor belt passes through a heated roller system (16) having a temperature of about 175-250° F. The number of heated rollers and their respective diameters and rotational speeds are configured and adjusted to match the timing of the previous processes, such that the combined process yields about 10 minutes of heating time to cure the LSR and remove the xylene solvent. The tension of the heated rollers is controlled and adjusted as needed to ensure the polyester fabric is in contact with the LSR layer. The cured polyester reinforced LSR composite film has a thickness of about 25 μm in the regions of LSR between the individual polyester reinforcing fibers.

At the 6th process station the cured reinforced LSR composite film (17) is separated from the conveyor belt (1) and cut into sheets by passing the film through a roller die (18) and a backing roller (19). Before depositing the cut sheets (21) back onto the conveyor belt, a roller mechanism (20) alternates the side of the sheet that is facing up when placed back onto the conveyor belt by engaging or disengaging the backing roller (19) as indicated by the arrow. If an optional carrier film is utilized, it is removed from the cut sheet at this time.

A silicone High Consistency Rubber (HCR) frame having an integrated tubing port is fed from a cassette feeder (22) and placed on top of the LSR sheets having the silicone surface facing up. The cut reinforced LSR sheet that has the polyester side facing up is lifted from the conveyor belt and placed on top of the sheet with the silicone HCR frame by a robotic arm (23) to yield a three-layer bag structure having two LSR sheets and one HCR frame.

The three-layer bag structures are then pressed together on the conveyor belt by a series of heated rollers (24), such that the distance between the heated rollers and the conveyor belt are maintained at a specified distance to provide slight compression to only the HCR frame, and to provide a curing cycle of about 10 minutes at 200-250° F. Optionally, the series of heated rollers may provide for incrementally increasing compression to ensure a leak-free seal of the LSR to HCR frame.

The cured three-layer collapsible blood containers (25) are transferred from the conveyor belt to a second conveyor belt system and second heat tunnel (not shown) for post-cure baking at about 230-250° F. for about 120-240 minutes. To maintain balance with the previous process steps the second heat tunnel may utilize a cassette loader system to be able to accept multiple collapsible blood container structures before entering the second heat tunnel, wherein the number of collapsible blood container structures within a cassette tray balances the rate of the heat tunnel to the previous process steps.

The post-baked collapsible blood containers are transferred from the heat tunnel to a turntable (not shown) for cooling to ambient temperature (about 10 minutes) before removing the collapsible blood containers from the cassette holders. Alternatively, the cooled cassette holders are transferred with the collapsible blood containers for further assembly and packaging.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

### Example 9: Fabrication of Silicone Reinforced Silicone Membranes 700 Suitable for Use in an Inner Collapsible Blood Container 102 Using a Compression Method

Plates are prepared having features 701 for compression molding of silicone reinforced silicone membranes 700 (FIG. 11A). A compression molded silicone membrane 700 has reinforcing features 701 in between open areas of the silicone membrane 702 arranged in a pattern 722. A membrane is made by depositing LSR on one plate and compressing it against the other plate set at a distance of 30 μm apart to make a membrane 30 μm thick. After compression, the resulting membrane is heated to 105° C. for 10 minutes until set.

### Example 10: Application of Reinforcement Features to a Preformed Silicone Membrane 113 Suitable for Use in an Inner Collapsible Blood Container 102

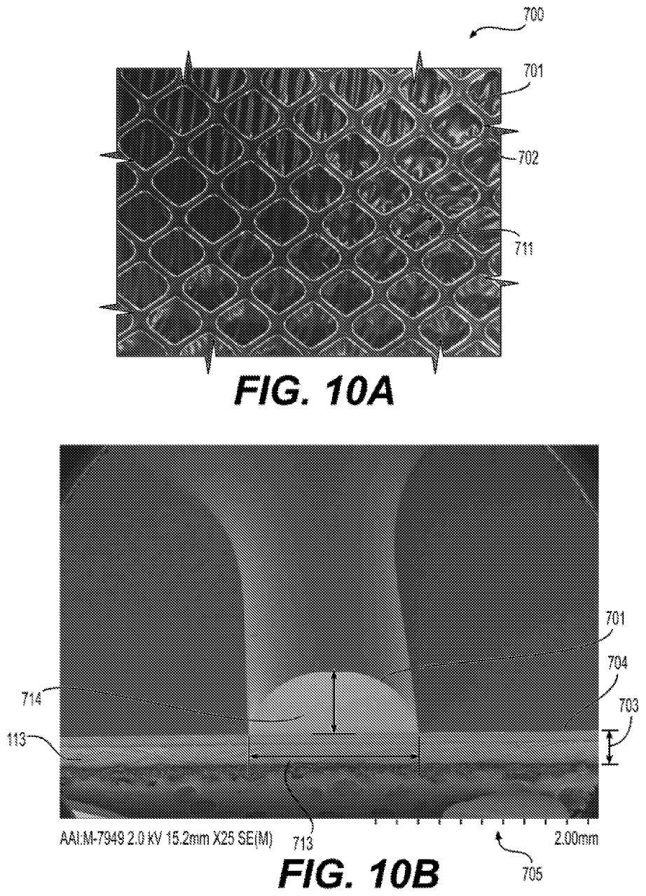

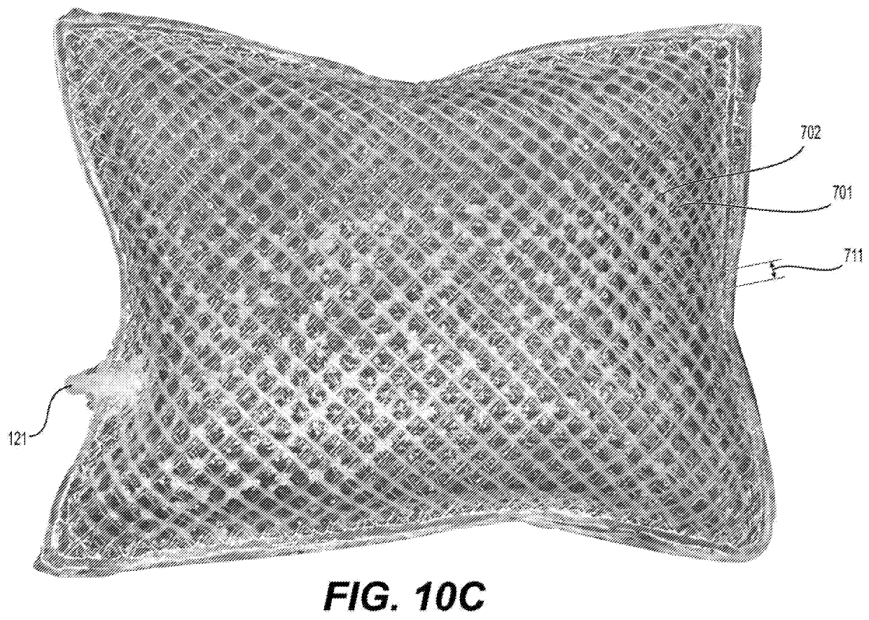

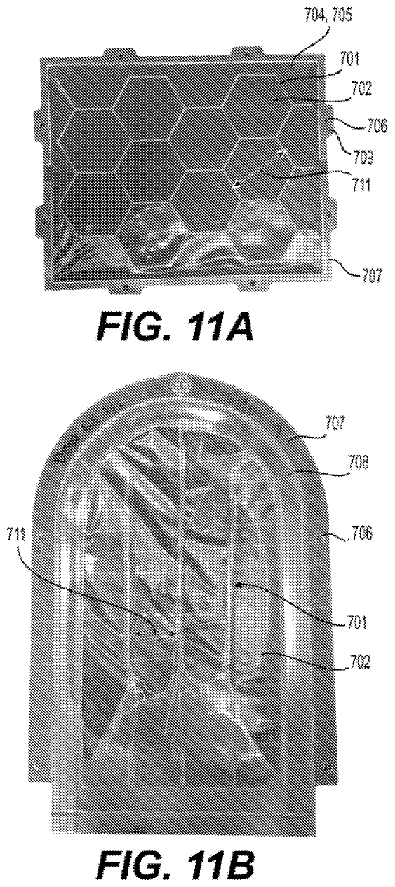

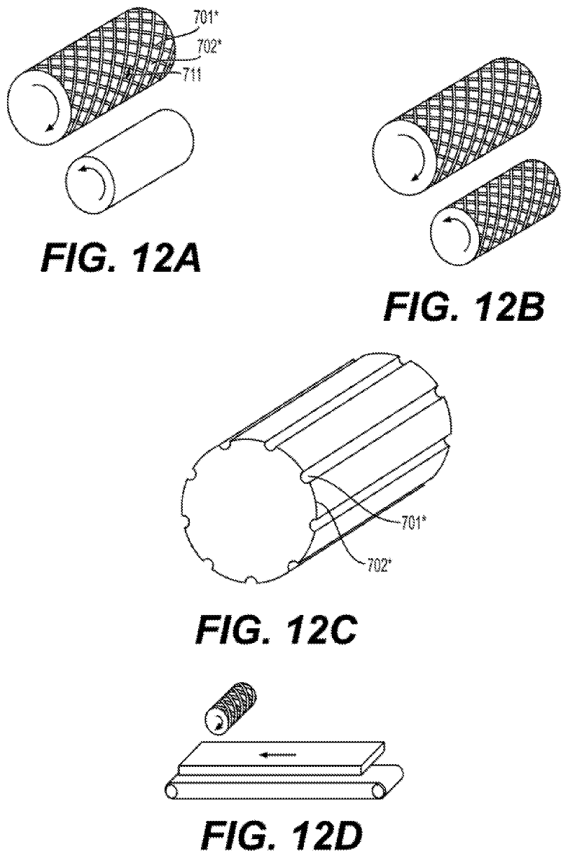

A silicone membrane 113 is prepared using a knife coating technique as described in Example 2. After partial curing, 2-part LSR is prepared as described in Example 2 and applied with an 18 gauge plastic tip connected to a 30 CC syringe creating raised feature 701 arranged in a pattern 721. The reinforcing raised features 701 have height 714, base width 713, and top width 715 and are separated by open areas 702 with an interstitial distance 711. After LSR application, the reinforced membrane 700 is cured in an oven at 105° C. for 10 minutes until set. A membrane 700 prepared according to this method is shown in FIG. 10A.

### Example 11: Fabrication of an Inner Collapsible Blood Container 102 Using Silicone Reinforced Silicone Membranes 700

### A) LSR Joining Method

Two reinforced silicone membranes 700, fabricated according to Example 10, are joined to prepare an inner collapsible blood container 102. An integrated frame 120 is created by tracing the edge of a reinforced membrane 700 with 2-part LSR using a 14 gauge plastic tip connected to a 30 CC syringe on the blood contact side of the membrane. The features 701 are arranged on the exterior surface of the container 102. Silicone tubing is placed on top of the frame in the desired location to form a fluidic connection 121 to the lumen of the container. A second reinforced silicone membrane 700 is joined to the first by the frame and the assembly is cured in an oven at 105° C. for 10 minutes until set. A container 102 prepared according to this method is shown in FIG. 10C.

### B) HCR Joining Method

Two reinforced silicone membranes 700 are joined by a die-cut frame 120 of HCR applied to the outer edge of a membrane. Space is left within the frame for placement of a silicone tube in the desired location to form a fluidic connection 121 to the lumen of the container. A second reinforced silicone membrane 700 is placed on top of the assembly, pressed to join, and cured in an oven at 105° C. for 10 minutes until set.

### C) Injection Molding Method

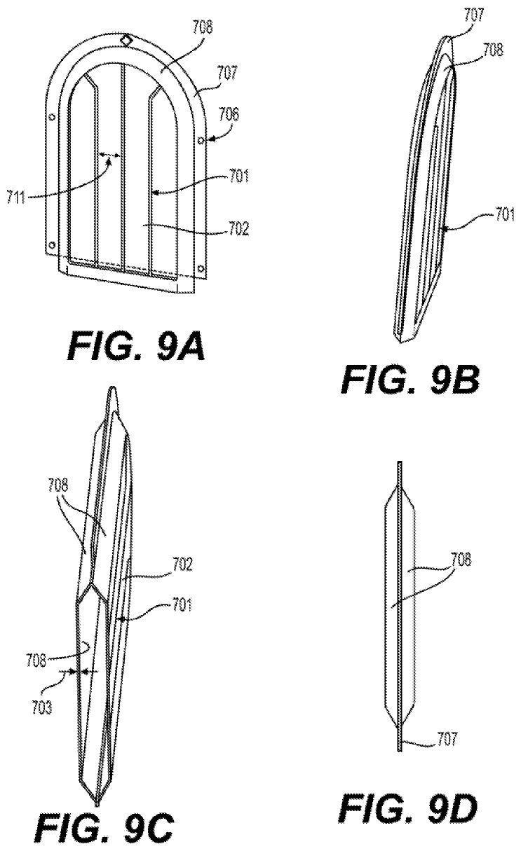

A collapsible blood container 102 is prepared using an injection mold prepared having a recessed feature 701*. The mold is used to make a silicone container 102 comprised of silicone reinforced silicone membranes 700 with raised feature 701 open on one side in a single process (FIGS. 9A to 9D and 11B). After a removable core is inserted into the mold, LSR is prepared as described in Example 2 and injected into the mold under pressure. The silicone membranes have a thickness equivalent to the space between the cavity and the core. The container, as shown in FIG. 11B, is cured in an oven at 105° C. for 10 minutes until set. Silicone tubing is placed in the desired location to form a fluidic connection 121 to the lumen of the container and the open end is then sealed with LSR, HCR or a thermoplastic clip.

### Example 12: Testing Peak Force Deflection Over Time

Low-cycle fatigue testing is performed in accordance with a modified ASTM E606-12 method (“Standard Test Method for Strain-Controlled Fatigue Testing,” Element Materials Technology) using a MTS Sintech Qtest compression testing machine (S/N 022197, MTS Systems Corp.) in conjunction with an actuator (S/N P16-150-22-12-9) and software (Firgelli Technologies). Flexibility of silicone membranes is tested by mounting a 12.7 mm diameter membrane to a fixture having a central test area of 7.6 mm in diameter. The test membrane is deflected 12.7 mm using a 2.54 mm spherical nozzle cycled at 1.2 hertz. The peak load at maximum deflection of each membrane is measured using a strain gauge attached to the spherical nozzle.

The peak load is determined every 5 minutes for 90 minutes during testing. The peak load values over the testing period are plotted and the average for each sample is obtained. Table 2 presents the results of 30 μm thick silicone membranes 113 as described in Example 10, fabric reinforced 25 μm silicone membranes 600 as described in Example 2, fabric reinforced 25 μm silicone membranes 600 that have been postcured and 30 μm silicone reinforced silicone membranes 700 as described in Example 11.

TABLE 3
Average Peak load over 90 minutes

| | | silicone | |
|---|---|---|---|
| silicone | silicone | membrane | silicone |
| membrane | membrane | 600, 25 μm | membrane |
| 113, 30 μm | 600, 25 μm | postcured | 70030 μm |
| 0.14 N | 6.82 N | 5.10 N | 0.39 N g |

### Example 13: Oxygen Depletion Testing Using Oxygen Depletion Devices Containing Reinforced Silicone Membranes

ORBs are prepared for oxygen depletion testing as described in Example 6 with inner collapsible blood containers 102 composed of 30 μm silicone membranes 113, fabric reinforced 25 μm silicone membranes 600, internally reinforced 30 μm silicone membranes 700, and externally reinforced 30 μm silicone membranes 700.

Oxygen depletion testing is carried out as described in Example 6. All experiments begin with ≥90% SO₂ prior to transferring the blood to an ORB and placing on a platelet shaker for 90 minutes. The outer receptacle 101 headspace oxygen level is measured using a Mocon OpTech® Platinum oxygen analyzer (MOCON, Inc., Minneapolis, MN). The kinetic rate and estimated final SO₂ concentration after 90 minutes is determined, the results are shown in Table 4.

TABLE 4
Permeability of inner collapsible blood containers 102

| | | fabric reinforced | internally reinforced | externally reinforced |
|---|---|---|---|---|
| | 30 μm silicone | 25 μm silicone | 30 μm silicone | 30 μm silicone |
| | membrane, 113 | membrane, 600 | membrane, 700 | membrane, 700 |
| Kinetic rate | −1.75 | −1.2 | −1.1 | −1.25 |
| (×10⁻² min⁻¹) | | | | |
| Estimated % S0₂ | 5.5% | 10% | 11.5% | 13.5% |
| after 90 min | | | | |
| n | 12 | 6 | 2 | 2 |

### Example 14: Permeability Testing of Reinforced Silicone Membranes

Permeability to O₂ diffusion is tested using the MOCON permeability test system according to manufacturer's instructions (MOCON, Inc). In this system, a MOCON Optech® O₂ Platinum analyzer is attached to a permeability test chamber and monitored using Optech® O₂ Platinum software. The membrane sample is placed in the chamber with a Tyvek® sheet on top and the chamber is sealed with the clamp. The tested is performed after the chamber is flushed with CO₂ until the partial pressure of O₂ stabilizes around 0.5-0.6 Torr.

Four membranes each of 30 μm silicone membranes 113, fabric reinforced 25 μm silicone membranes 600, and silicone reinforced 30 μm silicone membranes 700 are evaluated for 45 minutes. No detectable difference between the membranes are found. The permeability coefficient for 30 μm silicone membranes 113, fabric reinforced 25 μm silicone membranes 600, and silicone reinforced 30 μm silicone membranes 700 are all approximately 1.24×10⁻⁶ (ml*sec⁻¹*cm⁻²mmHg⁻¹).

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. An oxygen depletion device 10 for depleting oxygen from blood prior to anaerobic storage comprising:
an outer receptacle 101 substantially impermeable to oxygen;
an inner collapsible blood container 102 comprising one or more chambers that are permeable to oxygen; and
an oxygen sorbent 103 situated within said outer receptacle 101.

2. The oxygen depletion device of claim 1, wherein said an inner collapsible blood container 102 comprises a reinforced silicone membrane 600.

3. The oxygen depletion device of claim 2, wherein said oxygen depletion device 10 further comprises a headspace defined by said collapsible blood container 102 and said an outer receptacle 101 substantially impermeable to oxygen, wherein said oxygen sorbent 103 is disposed.

4. A silicone membrane 700 comprising:
a silicone membrane 113 having an area 702 and an average thickness 703 of less than 100×10⁻⁶ M (μm), and a feature 701 on at least one side of said silicone membrane 113 comprising silicone having an average length 713 of between 100 μm and 10000 μm perpendicular to an average length 714 of between 20 μm and 5000 μm.

5. The silicone membrane 700 of claim 4, wherein said raised feature 701 covers a percentage of less than 50% of the area of said at least one side of said silicone membrane 700.

6. The silicone membrane 700 of claim 4, wherein said raised feature 701 comprises a cross-section 760 selected from the group consisting of:
a. cross-section 761 comprising a length 714 of between 100 μm and 2500 μm and a radius of length 718 of between 100 μm and 500 μm;
b. cross-section 762 comprising a length 713 of between 100 μm and 2500 μm, a length 714 of between 100 μm and 2500 μm, and a length 715 of zero;
c. cross-section 762 comprising a length 713 of between 100 μm and 2500 μm, a length 714 of between 100 μm and 2500 μm, wherein length 715 equals length 713;
d. cross-section 762 comprising a length 713 of between 100 μm and 2500 μm, a length 714 of between 100 μm and 2500 μm, wherein length 715 is greater than length 713;
e. cross-section 763 comprising a length 713 of between 100 μm and 2500 μm, a length 714 of between 100 μm and 2500 μm, and a radius of length 718 between 5 μm and 100 wherein length 715 is equal to length 713;
f. cross-section 764 comprising a length 713 of between 100 μm and 2500 μm, a length 714 of between 100 μm and 2500 μm, and an angle 719 between 20° and 60°, wherein length 715 is equal to length 713;
g. cross-section 765 comprising a length 713 of between 100 μm and 2500 μm, a length 714 of between 100 μm and 2500 μm, and a radius of length 718 between 5 μm and 100 μm, wherein length 715 is equal to length 713;
h. cross-section 766 comprising a length 713 of between 100 μm and 2500 μm, a length 714 of between 100 μm and 2500 μm, and an angle 719 of between 20° and 60°; and
i. cross-section 767 comprising a length 713 of between 100 μm and 2500 μm, a length 714 of between 100 μm and 2500 μm, and a radius of length 718 of between 5 μm and 100 μm or between 15 μm and 30 μm and a length 715 of 50 to 0 μm.

7. The silicone membrane 700 of claim 4, wherein said raised feature 701 comprises a pattern 720 selected from the group consisting of
(i) pattern 721, wherein length 711 is between 2 mm and 72 mm, and length 714 is between 4.0 mm and 72 mm;
(ii) pattern 722, wherein length 711 is between 2 mm and 72 mm;
(iii) pattern 723 wherein radius 718 is between 2 mm and 72 mm;
(iv) pattern 724, wherein length 711 is between 2 mm and 72 mm;
(v) pattern 725, wherein length 711 is between 2 mm and 72 mm, wavelength 716 is between 5 mm and 200 mm, and amplitude 717 is between 5 and 72 mm;
(vi) pattern 726, wherein unit cell 710 has length 711 is between 2 mm and 72 mm
(vii) pattern 727, wherein length 711 is between 2 mm and 72 mm and length 712 is between 2 mm and 72 mm;
(viii) pattern 728, wherein length 711 is between 2 mm and 72 mm and length 712 is between 2 mm and 72 mm, and angle 719 is less than 90°;
(ix) pattern 728, wherein length 711 is between 2 mm and 72 mm and length 712 is between 2 mm and 72 mm, and angle 719 is 90°;
(x) pattern 729, wherein length 711 is between 2 mm and 72 mm and length 712 is between 2 mm and 72 mm;
(xi) pattern 730, wherein unit cell 710 has length 711 is between 2 mm and 72 mm;
(xii) pattern 731, wherein unit cell 710 has length 711 is between 2 mm and 72 mm and length 712 is between 2 mm and 72 mm;
(xiii) pattern 732, wherein unit cell 710 has length 711 is between 2 mm and 72 mm and length 712 is between 2 mm and 72 mm;
(xiv) pattern 733, wherein length 711 is between 2 mm and 72 mm and is the average length of features 701.

8. The silicone membrane 700 of claim 4, wherein said silicone membrane 700 has a permeability of at least 1.2×10⁻⁶ ml/sec*cm²*mmHg.

9. An inner collapsible blood container 102 comprising a silicone membrane 700 according claim 4.

10. (canceled)

11. (canceled)

12. A method of manufacturing a silicone membrane 700 comprising
(i) preparing silicone membrane 113 having a thickness of less than 100×10⁻⁶ M (μm);
(ii) applying a raised feature 701 having an average length 713 of between 100 μm and 10000 μm perpendicular to an average length 714 of between 20 μm and 5000 μm to the surface of said silicone membrane 113; and
(iii) curing said silicone membrane 700.

13. The method of manufacturing a silicone membrane 700 of claim 12, wherein said preparing of step (i) and said applying of step (ii) are a continuous process.

14. (canceled)
